SEQUENCE LISTING (39 sequences)

SEQ ID NO: 1 - protein, 107 aa, Artificial sequence
  Sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 2 - protein, 122 aa, Artificial sequence [sequence omitted]

SEQ ID NO: 3 - protein, 213 aa, Artificial sequence
  Sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 452 aa, Artificial sequence
  Sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 452 aa, Artificial sequence
  Sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 250 aa, Mus musculus
MKKNIAFLLASMFVFSIATNAYAQVQLQQSGAELAKPGASVKMSCKASGYNFTTYWMHWVKQRPGQGLEWIGYINPSTDYTEYNQKFKYKATLTADKSSSTAYIQLSSLTSEDSAVYYCARWWDYDWYFDVWGAGTPLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 7 - protein, 227 aa, Mus musculus
QVQLQQSGAELAKPGASVKMSCKASGYNFTTYWMHWVKQRPGQGLEWIGYINPSTDYTEYNQKFKYKATLTADKSSSTAYIQLSSLTSEDSAVYYCARWWDYDWYFDVWGAGTPLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 8 - protein, 237 aa, Mus musculus
MKKNIAFLLASMFVFSIATNAYADIVMTQSQEFMSTSVGDRVSVTCKASQTVDTNVAWYQQKLGQSPKPLIYSASYRCSGVPDRFTGSGSRTDFTLTITNVQSEDLAEYFCQQYHSFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 214 aa, Mus musculus
DIVMTQSQEFMSTSVGDRVSVTCKASQTVDTNVAWYQQKLGQSPKPLIYSASYRCSGVPDRFTGSGSRTDFTLTITNVQSEDLAEYFCQQYHSFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - DNA, 1701 bp, Mus musculus
  Unsure 1684: Unknown amino acid
actagtacgcaagttcacgtaaaaagggtatctagaattatgaagaagaatattgcgttcctacttgcctctatgtttgtcttttctatagctacaaacgcgtatgctgatatcgtgatgacccagtctcaagaattcatgtccacatcagtaggagacagggtcagcgtcacctgcaaggccagtcagactgtggatactaatgtagcctggtatcaacagaaactagggcaatctcctaaaccactgatttactcggcatcctaccggtgtagtggggtccctgatcgcttcacaggcagtggatctcggacagatttcactctcaccatcaccaatgtgcagtctgaagacttggcagagtatttctgtcagcaatatcacagttttccgtggacgttcggtggaggtaccaaggtggagatcaaacgaactgtggctgcaccatctgtcttcatcttcccgccatctgatgagcagttgaaatctggaactgcttctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaagctgatcctctacgccggacgcatcgtggccctagtacgcaactagtcgtaaaaagggtatctagaggttgaggtgattttatgaaaaagaatatcgcatttcttcttgcatctatgttcgttttttctattgctacaaacgcgtacgctcaggttcagctgcagcagtctggggctgaactggcaaaacctggggcctcagtgaagatgtcctgcaaggcttctggctacaactttactacctactggatgcactgggtaaaacagaggcctggacagggtctggaatggattggatacattaatcctagcactgattatactgagtacaatcagaagttcaagtacaaggccacattgactgcagacaaatcctccagcacagcctacattcaactgagcagcctgacatctgaggactctgcagtctattactgtgcaagatggtgggattacgactggtacttcgatgtctggggcgcagggaccccactcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacataaccaccgcatgcgacggccctagagtccctaacgctcggttgccgccgggcgtttttttattgttaactcatgtttgacagcttatcatmgataaacntttatgcggtagttatc

SEQ ID NO: 11 - protein, 122 aa, Mus musculus
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSS

SEQ ID NO: 12 - protein, 122 aa, Artificial sequence [sequence omitted]

SEQ ID NO: 13 - protein, 119 aa, Artificial sequence
  Sequence is synthesized
EVQLVESGGGLVQPGGSLRLSCAASGFTFSSYAMSWVRQAPGKGLEWVAVISGDGGSTYYADSVKGRFTISRDNSKNTLYLQMNSLRAEDTAVYYCARGRVGYSLYDYWGQGTLVTVSS

SEQ ID NO: 14 - protein, 10 aa, Mus musculus
GYTFTSYNMH

SEQ ID NO: 15 - protein, 17 aa, Artificial sequence
  Sequence is synthesized
AIYPGNGDTSYNQKFKG

SEQ ID NO: 16 - protein, 13 aa, Artificial sequence
  Sequence is synthesized
VVYYSNSYWYFDV

SEQ ID NO: 17 - protein, 107 aa, Mus musculus
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKR

SEQ ID NO: 18 - protein, 107 aa, Artificial sequence; identical to SEQ ID NO: 1
  Sequence is synthesized

SEQ ID NO: 19 - protein, 108 aa, Artificial sequence
  Sequence is synthesized
DIQMTQSPSSLSASVGDRVTITCRASQSISNYLAWYQQKPGKAPKLLIYAASSLESGVPSRFSGSGSGTDFTLTISSLQPEDFATYYCQQYNSLPWTFGQGTKVEIKR

SEQ ID NO: 20 - protein, 10 aa, Mus musculus
RASSSVSYMH

SEQ ID NO: 21 - protein, 7 aa, Artificial sequence
  Sequence is synthesized
APSNLAS

SEQ ID NO: 22 - protein, 9 aa, Artificial sequence
  Sequence is synthesized
QQWSFNPPT

SEQ ID NO: 23 - protein, 232 aa, Artificial sequence
  Sequence is synthesized
MGWSCIILFLVATATGVHSDIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 213 aa, Artificial sequence; identical to SEQ ID NO: 3
  Sequence is synthesized

SEQ ID NO: 25 - protein, 471 aa, Artificial sequence
  Sequence is synthesized
MGWSCIILFLVATATGVHSEVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 452 aa, Artificial sequence; identical to SEQ ID NO: 4
  Sequence is synthesized

SEQ ID NO: 27 - protein, 471 aa, Artificial sequence
  Sequence is synthesized
MGWSCIILFLVATATGVHSEVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 452 aa, Artificial sequence; identical to SEQ ID NO: 5
  Sequence is synthesized

SEQ ID NO: 29 - protein, 10 aa, Artificial sequence
  Sequence is synthesized
  Xaa 9: X = M or L
RASSSVSYXH

SEQ ID NO: 30 - protein, 9 aa, Artificial sequence
  Sequence is synthesized
  Xaa 4: X = S or A
QQWXFNPPT

SEQ ID NO: 31 - protein, 17 aa, Artificial sequence
  Sequence is synthesized
  Xaa 8: X = D or A
AIYPGNGXTSYNQKFKG

SEQ ID NO: 32 - protein, 13 aa, Artificial sequence
  Sequence is synthesized
  Xaa 6: X = N, A or Y
  Xaa 7: X = S or R
VVYYSXXYWYFDV

SEQ ID NO: 33 - protein, 213 aa, Artificial sequence
  Sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 452 aa, Artificial sequence
  Sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 213 aa, Artificial sequence; identical to SEQ ID NO: 33
  Sequence is synthesized

SEQ ID NO: 36 - protein, 452 aa, Artificial sequence; identical to SEQ ID NO: 34
  Sequence is synthesized

SEQ ID NO: 37 - protein, 107 aa, Artificial sequence
  Sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKR

SEQ ID NO: 38 - protein, 213 aa, Artificial sequence; identical to SEQ ID NO: 33
  Sequence is synthesized

SEQ ID NO: 39 - protein, 452 aa, Artificial sequence
  Sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2008/0176257 A9
(43) Pub. Date: Jul. 24, 2008

(54) Assay for Antibodies

(75) Inventors: Anan Chuntharapai, Colma, CA (US); Kyu H. Hong, Newark, CA (US); Yu-Ju G. Meng, Albany, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 11/535,422

(22) Filed: Sep. 26, 2006

Related U.S. Application Data
(63) Continuation of application No. 11/106,762, filed on Apr. 15, 2005.
(60) Provisional application No. 60/563,193, filed on Apr. 16, 2004.

(51) Int. Cl. G01N 33/53; C07K 16/18; C12N 5/06

(52) U.S. Cl. 435/7.92; 530/388.1; 530/391.1; 435/326

(57) ABSTRACT

The presence and quantity of an antibody of interest in a patient's bloodstream or other biological sample can serve as an important clinical or other analytical or diagnostic tool. ELISA methods, and kits for such assays, as well as anti-idiotypic antibodies and hybridomas producing them, are developed to detect levels of the antibody in biological samples, which are from, for example, animal models and human patients.

### RELATED APPLICATIONS

This application is a continuation application of Ser. No. 11/106,762 filed on Apr. 15, 2005, which application claims priority to and the benefit of U.S. provisional application Ser. No. 60/563193 filed Apr. 16, 2004, the contents of which are incorporated herein by reference.

### FIELD OF THE INVENTION

The present invention relates to a high-throughput assay based on use of anti-idiotypic antibodies for detecting antibodies to transmembrane antigens with small extracellular domains, such as for quantitating humanized anti-CD20 antibody in serum for clinical studies.

### BACKGROUND OF THE INVENTION

Transmembrane proteins extend through the lipid bilayer, with part of their mass on either side, having regions that are hydrophobic and regions that are hydrophilic. Typically, a transmembrane protein has its cytoplasmic domain and extracellular domain, which are separated by the membrane-spanning segments of the polypeptide chain. The membrane-spanning segments contact the hydrophobic environment of the lipid bilayer and are composed largely of amino acid residues with non-polar side chains. The great majority of transmembrane proteins are glycosylated. The oligosaccharide chains are usually present in the extracellular domain. Further, the reducing environment of the cytosol prevents the formation of intrachain (and interchain) disulfide (S—S) bonds between cysteine residues on the cytosolic side membranes. These disulfide bonds do form on the extracellular side, e.g., between the N-terminal domain and an extracellular domain.

Transmembrane proteins are notoriously difficult to crystallize for X-ray structural studies. The folded three-dimensional structures are quite uncertain for the isolated forms of these proteins. Thus, these features present a problem in the attempt to use the whole transmembrane protein as a target for isolating molecules that would bind to it in vitro.

G-protein-coupled receptors (GPCR) are a superfamily of transmembrane proteins that play important roles in the signal-transduction process of a cell. GPCR mediate the cellular responses to an enormous diversity of signaling molecules, including hormones, neurotransmitters, and local mediators. The signal molecules vary in their structure and function, including proteins and small peptides, as well as amino acid and fatty acid derivatives. See reviews by Watson and Arkinstall, The G-Protein Linked Receptor Facts Book (Academic Press, Harcourt Brace & Company, Publishers, London, San Diego, New York: 1994); Proudfoot et al., Nature Review Immunology, 2:106-115 (2002); and Ji et al., J. Biol. Chem., 273:17299-17302 (1998)).

For example, receptors for the hormone relaxin (LGR7 and LGR8) have been found recently to be G-protein coupled receptors (Hsu et al., Science, 295:671-674 (2002)). Relaxin is a hormone important for the growth and remodeling of reproductive and other tissues during pregnancy. Hsu et al. demonstrated that two orphan heterotrimeric guanine nucleotide binding protein (G-protein) receptors, LGR7 and LGR8, are capable of mediating the action of relaxin through an adenosine 3′,5′-monophosphate (cAMP)—dependent pathway distinct from that of the structurally related insulin and insulin-like growth factor. These receptors for relaxin are implicated to play roles in reproductive, brain, renal, cardiovascular, and other functions.

Despite the chemical and functional diversity of the signaling molecules that bind to them, all of GPCRs share a structural similarity in that the polypeptide chain threads back and forth across the lipid bilayer several times, e.g., seven times to form seven transmembrane domains that are connected by three extracellular loops and three intracellular loops.

Both CCR5 and CXCR4 are chemokine receptors that are members of the GPCR superfamily. CCR5 is a receptor for several CC chemokines such as MIP-1α(also named GOS19, LD78, pAT464 gene product, TY5 (murine) and SISα(murine)), MIP-1β(also named Act-2, G-26, pAT744 gene product, H-400 (murine) and hSISγ(murine)), and RANTES (regulated on activation, normal T cell expressed and secreted, or CCL5) (Cocchi et al., Science, 270:1811-1815 (1995) and Mellado et al., Annu. Rev. Immunol., 19:397-421 (2001)). CXCR4 (also named LESTR or fusin before) is a human chemokine receptor with the C—X—C motif, and is highly expressed in leukocytes (Loetscher et al., J. Biol. Chem., 269:232-237 (1994)). The lymphocyte chemoattractant stromal cell derived factor-1 (or SDF-1) or CXCL12 is a ligand for CXCR4 (Bleul et al., Nature, 382:829-833 (1996)). CXCR4 acts as a co-receptor of HIV-1 (Feng, Science, 272:872-877 (1996)). Its expression is also correlated with cancer, including prostate cancer (Taichman et al., Cancer Res., 62:1832-1837 (2002)) and breast cancer metastasis (Muller et al ., Nature, 410:50-56 (2001) and Moore, Bioessays, 23:674-676 (2001)). The antibodies generated from these chemokine receptors can then be used for the prevention and/or treatment of HIV infection, cancer, and other diseases associated with abnormal chemokine activities. Human monoclonal single-chain antibodies against CCR5 and CXCR4 can be used to inhibit HIV infection of peripheral blood mononuclear cells and chemotaxis in breast cancer cells, respectively.

The amino acid sequence of human CCR5 has seven transmembrane domains that are connected by loops 2, 4, and 6, which are extracellular loops, and by loops 1, 3, and 5, which are intracellular loops. A model of the secondary structure of human CCR5 is provided in Blanpain et al., J. Biol. Chem., 274:34719-34727 (1999).

Other than CCR5 and CXCR4, examples of a chemokine receptor or a chemokine receptor-like orphan receptor also include, but are not limited to, CCR1, CCR2b, CCR3, CCR4, CCR8, CXCR1, CXCR2, CXCR3, CX3CR1, STRL33/BONZO, and GPR15/BOB (Berger et al., AIDS, 11, Suppl. a: S3-S16 (1997) and Dimitrov, Cell, 91: 721-730 (1997)). Each or a set of these HIV co-receptors can mediate entry of different strains of HIV virus into the host cell.

The chemokine superfamily comprises two main branches: the α-chemokines (or CXC chemokines) and the β-chemokines (CC chemokines). The α-chemokine branch includes proteins such as IL-8, neutrophil-activating peptide-2 (NAP-2), melanoma growth stimulatory activity (MGSA/gro or GROA), and ENA-78, each of which have attracting and activating effects predominantly on neutrophils. The members of the β-chemokine branch affect other cell types such as monocytes, lymphocytes, basophils, and eosinophils (Oppenheim et al., Annu. Rev. Immunol., 9:617-648 (1991); Baggiolini et al., Adv. Imunol., 55:97-179 (1994); Miller and Krangel, Crit. Rev. Immunol., 12:17-46 (1992); Jose et al., J. Exp. Med., 179:881-118 (1994); Ponath et al., J. Clin. Invest., 97:604-612 (1996)), and include proteins such as monocyte chemotactic proteins 1-4 (MCP-1, MCP-2, MCP-3, and MCP-4), RANTES, and macrophage inflammatory proteins (MIP-1α, MIP-1β). Recently, a new class of membrane-bound chemokines designated CX3C chemokines has been identified (Bazan et al., Nature, 385:640-644 (1997)). Chemokines can mediate a range of pro-inflammatory effects on leukocytes, such as triggering of chemotaxis, degranulation, synthesis of lipid mediators, and integrin activation (Oppenheim et al., Annu. Rev. Immunol., 9:617-648 (1991); Baggiolini et al., Adv. Imunol., 55:97-179 (1994); Miller and Krangel, Crit. Rev. Immunol., 12:17-46 (1992)). Lately, certain β-chemokines have been shown to suppress HIV-1 infection of human T-cell lines in vitro (Cocchi et al., Science, 270:1811-1815 (1995)).

Chemokines bind to seven transmembrane-spanning (7TMS) G protein-coupled receptors (Murphy, Annu. Rev. Immunol., 12:593-633 (1994)). Some known receptors for the CC or β chemokines include CCR1, which binds MIP-1α and RANTES (Neote et al., Cell, 72:415-425 (1993); Gao, J. Exp. Med., 177:1421-1427 (1993)); CCR2, which binds chemokines including MCP-1, MCP-2, MCP-3 and MCP-4 (Charo et al., Proc. Natl. Acad. Sci. USA, 91:2752-2756 (1994); Myers et al., J. Biol. Chem., 270:5786-5792 (1995); Gong et al., J. Biol. Chem., 272:11682-11685 (1997); Garcia-Zepeda et al., J. Immunol., 157:5613-5626 (1996)); CCR3, which binds chemokines including eotaxin, RANTES and MCP-3 (Ponath et al., J. Exp. Med., 183:2437-2448 (1996)); CCR4, which has been found to signal in response to MCP-1, MIP-1α, and RANTES (Power et al., J. Biol. Chem., 270:19495-19500 (1995)); and CCR5, which has been shown to signal in response to MIP-1α, MIP-1β, and RANTES (Boring et al., J. Biol. Chem., 271 (13):7551-7558 (1996); Raport, J. Biol. Chem., 271:17161-17166 (1996); and Samson et al., Biochemistry, 35:3362-3367 (1996)).

CCR2 is expressed on the surface of several leukocyte subsets, and appears to be expressed in two slightly different forms (CCR2a and CCR2b) due to alternative splicing of the mRNA encoding the carboxy-terminal region (Charo et al., Proc. Natl. Acad. Sci. USA, 91:2752-2756 (1994)). MCP-1 acts upon monocytes, lymphocytes, and basophils, inducing chemotaxis, granule release, respiratory burst, and histamine and cytokine release. Studies have suggested that MCP-1 is implicated in the pathology of diseases such as rheumatoid arthritis, atherosclerosis, granulomatous diseases, and multiple sclerosis (Koch, J. Clin. Invest., 90:772-79 (1992); Hosaka et al., Clin. Exp. Immunol., 97:451-457 (1994); Schwartz et al., Am. J. Cardiol., 71(6):9B-14B (1993); Schimmer et al., J. Immunol., 160:1466-1471 (1998); Flory et al., Lab. Invest., 69:396-404 (1993); Gong et al., J. Exp. Med., 186:131- 137 (1997)). Additionally, CCR2 can act as a co-receptor for HIV (Connor et al., J. Exp. Med., 185:621-628 (1997)). Thus, CCR2 receptor antagonists may represent a new class of important therapeutic agents.

CD20 is a 33-36-kDa non-glycosylated membrane protein that exists as different alternate splicing variants on normal and malignant B cells. It has four membrane-spanning hydrophobic regions with intracellular termini and a short intervening extracellular loop of about 42 amino acids (Tedder et al., Proc. Natl. Acad. Sci. USA, 85: 208-212 (1988); Einfeld et al., EMBO, 7: 711-717 (1988)). A chimeric anti-CD20 antibody, rituximab (RITUXAN®), has been used to deplete B cells in patients with non-Hodgkin's lymphoma as part of the standard therapy. It also has been efficacious in treating some autoimmune diseases (Boye et al., Annals of Oncology, 14: 520-535 (2003); Von Schilling et al., Seminars in Cancer Biology, 13: 211-222 (2003); Kneitz et al., Immunobiology, 206:519-527 (2002)). A humanized antibody is preferred for long-term treatment of B-cell-associated disorders since it is less likely to cause immune response (Boye et al., supra; Maeda et al., International Journal of Hematology, 74: 70-75 (2001)). However, the small extracellular loop of CD20, which is between two membrane-spanning regions, is difficult to express in its native conformation, as are many of the CXC-chemokine and CC-chemokine receptors. Typically, immunoassays for high-concentration, high-molecular-weight analytes in the marketplace are predicated on the multivalence of the analyte. Ultimately, the analyte is detected by some sort of cross-linking, either by agglutination (in turbidimetric or nephelometric assays), precipitation (radial immunodiffusion), or sandwich immunoassays such as ELISAs.

U.S. Pub. No. US 20020142356 provides a method for obtaining anti-idiotypic monoclonal antibody populations directed to an antibody that is specific for a high-concentration, high-molecular-weight target antigen wherein said anti-idiotypic antibody populations have a wide range of binding affinities for the selected antibody specific to said target antigen and wherein a subset of said anti-idiotypic antibody populations can be selected having the required affinity for a particular application. U.S. Pub. No. US 20020142356 involves a competitive immunoassay of an antigen using an antibody as coat and an anti-idiotypic antibody as detection or vice-versa. Other references disclosing use of an anti-idiotypic antibody as a surrogate antigen include Losman, Cancer Research, 55 (23 suppl S):S5978-S5982 (1995); Becker, J. of Immunol. Methods, 192 (1-2): 73-85 (1996); Baral, International J of Cancer, 92(1) 88-95 (2001); and Kohen, Food and Agriculture Immunology, 12(3) 193-201 (2000).

Enzyme-linked immunosorbent assays (ELISAs) for various antigens include those based on colorimetry, chemiluminescence, and fluorometry. ELISAs have been successfully applied in the determination of low amounts of drugs and other antigenic components in plasma and urine samples, involve no extraction steps, and are simple to carry out. ELISAs for the detection of antibodies to protein antigens often use direct binding of short synthetic peptides to the plastic surface of a microtitre plate. The peptides are, in general, very pure due to their synthetic nature and efficient purification methods using high-performance liquid chromatography. A drawback of short peptides is that they usually represent linear, but not conformational or discontinuous epitopes. To present conformational epitopes, either long peptides or the complete native protein is used. Direct binding of the protein antigens to the hydrophobic polystyrene support of the plate can result in partial or total denaturation of the bound protein and loss of conformational epitopes. Coating the plate with an antibody, which mediates the immobilization (capture ELISA) of the antigens, can avoid this effect. However, frequently, overexpressed recombinant proteins are insoluble and require purification under denaturing conditions and renaturation, when antibodies to conformational epitopes are to be analyzed. See, for example, U.S. Pub. No. US 20030044870 for a generic ELISA using recombinant fusion proteins as coat proteins.

Previously, cell-based ELISA methods using live suspension cells for screening hybridomas or for detecting antibodies against cell-surface antigens were reported (Posner et al., J. Immunol. Methods, 48: 23 (1982); Morris et al., Hum. Immunol., 5: 1 (1982); Grunow et al., J. Immunol. Meth., 171: 93 (1994)). Centrifugation was used for the wash steps. Simple cellular ELISA (CELISA) methods were also described (Sedgwick and Czerkinsky, J. Immunol. Meth., 150: 159 (1992)) using formaldehyde- or glutaraldehyde-fixed suspension (Walker et al., J. Immunol. Meth., 154: 121 (1992); Smith et al., BioTechniques, 22: 952 (1997); Yang et al., J. Immunol. Meth., 277:87 (2003)) or adherent cells (Smith et al., supra) as well as non-fixed dried cells (Arunachalam et al., J. Immunol. Meth., 135: 181 (1990); Schlosser et al. J. Immunol. Meth., 140: 101 (1991)) for detection of antibodies against cell-surface antigens or characterization of cell-surface molecules. Without use of live cells, there is a potential alteration of the epitope on CD20 caused by fixation or drying (Baron et al., Scand. J. Immunol., 6: 385 (1977), Schlosser et al., supra; Sedgwick and Czerkinsky, supra).

In addition, Meng et al., “Measuring CD20 binding for humanization of anti-CD20 antibody”, FASEB Journal, volume 18, No. 4, A59, program no. 85.8 (2004) discloses that an anti-idiotypic antibody specific to a humanized antibody can be used in an ELISA format to measure the serum concentrations of the antibody for clinical studies, but does not contain details. Hong et al., J. Immunol. Meth., 294: 189-197 (2004) discloses the quantitative live-cell and anti-idiotypic antibody-based ELISA for humanized antibody directed to CD20.

Since a soluble extracellular domain of many antigens such as CD20 and the chemokine receptors with the native conformation is not available as a capture reagent for measuring in selected samples the concentration of antibody binding to such domain, there is a need for measuring concentrations of antibodies that bind to such proteins. There is also a need to detect humanized antibodies to such cell-surface proteins in biological samples without also detecting certain other antibodies directed or not directed to such cell-surface proteins, particularly in clinical samples.

### SUMMARY OF THE INVENTION

Accordingly, the invention is as claimed. In one embodiment, an enzyme-linked immunosorbent assay (ELISA) method is provided for specifically detecting in a biological sample an antibody of interest that binds to a cell-surface, multi-transmembrane protein comprising an intervening extracellular domain of less than about 75 amino acids, which method comprises (a) contacting and incubating the biological sample with a capture reagent, wherein the capture reagent is an anti-idiotypic antibody binding to the idiotype of the antibody of interest but not to the idiotype of at least one other antibody in the sample that binds to the protein, so as to bind any of the antibody of interest present in the sample, and (b) contacting the sample, and hence any bound antibody of interest, with a detectable antibody that binds to the antibody of interest, and measuring the level of any of the antibody of interest bound to the capture reagent using a detection means for the detectable antibody. The capture reagent does not bind to the idiotype of at least one other antibody in the sample that binds to the protein so that the antibody of interest can be distinguished from such antibody or antibodies present in the sample. Preferably, the assay is cell based.

Preferably, the antibody of interest is a monoclonal antibody, more preferably a humanized antibody or murine antibody.

In another preferred embodiment, the detectable antibody is a detectable anti-idiotypic antibody binding to the idiotype of the antibody of interest but not to the idiotype of at least one other antibody in the sample that binds to the protein. The capture reagent and detectable antibody may be the same or different.

In another preferred aspect, the biological sample is isolated from a human subject or mouse subject. The biological sample is preferably plasma, serum, or urine, and most preferably serum.

Further preferred is the method wherein the measuring step further comprises using a standard curve to determine the level of the antibody of interest compared to a known level.

In another preferred aspect, the protein is CD20 and the antibody of interest is a humanized 2H7 antibody. Such humanized antibody of interest is preferably an intact antibody or antibody fragment comprising the variable light-chain sequence:

| | |
|---|---|
| DIQMTQSPSSLSASVGDRVTITCRASSSVSYMHWYQQKPGKAPKPLIYAPSNLASGVPSRFSG | (SEQ ID NO: 1) |
| SGSGTDFTLTISSLQPEDFATYYCQQWSFNPPTFGQGTKVEIKR; | |

and the variable heavy-chain sequence:

| | |
|---|---|
| EVQLVESGGGLVQPGGSLRLSCAASGYTFTSYNMHWVRQAPGKGLEWVGAIYPGNGDTSY | (SEQ ID NO: 2) |
| NQKFKGRFTISVDKSKNTLYLQMNSLRAEDTAVYYCARVVYYSNSYWYFDVWGQGTLVTV | |
| SS. | |

Where the humanized 2H7 antibody is an intact antibody, preferably it comprises the light-chain amino acid sequence:

| | |
|---|---|
| DIQMTQSPSSLSASVGDRVTITCRASSSVSYMHWYQQKPGKAPKPLIYAPSNLASGVPSRFSG | (SEQ ID NO: 3) |
| SGSGTDFTLTISSLQPEDFATYYCQQWSFNPPTFGQGTKVEIKRTVAAPSVFIFPPSDEQLKSG | |
| TASVVCLLNNFYPREAKVQWKVDNALQSGNSQESVTEQDSKDSTYSLSSTLTLSKADYEKH | |
| KVYACEVTHQGLSSPVTKSFNRGEC; | |

and the heavy-chain amino acid sequence:

| | |
|---|---|
| EVQLVESGGGLVQPGGSLRLSCAASGYTFTSYNMHWVRQAPGKGLEWVGAIYPGNGDTSY | (SEQ ID NO: 4) |
| NQKFKGRETISVDKSKNTLYLQMNSLRAEDTAVYYCARVVYYSNSYWYFDVWGQGTLVTV | |
| SSASTKGPSVFPLAPSSKSTSGGTAALGCLVKDYFPEPVTVSWNSGALTSGVHTFPAVLQSSG | |
| LYSLSSVVTVPSSSLGTQTYICNVNHKPSNTKVDKKVEPKSCDKTHTCPPCPAPELLGGPSVF | |
| LFPPKPKDTLMISRTPEVTCVVVDVSHEDPEVKFNWYVDGVEVHNAKTKPREEQYNSTYRV | |
| VSVLTVLHQDWLNGKEYKCKVSNKALPAPIEKTISKAKGQPREPQVYTLPPSREEMTKNQVS | |
| LTCLVKGFYPSDIAVEWESNGQPENNYKTTPPVLDSDGSFFLYSKLTVDKSRWQQGNVFSCS | |
| VMHEALHNHYTQKSLSLSPGK | |

or the heavy-chain amino acid sequence:

| | |
|---|---|
| EVQLVESGGGLVQPGGSLRLSCAASGYTFTSYNMHWVRQAPGKGLEWVGAIYPGNGDTSY | (SEQ ID NO: 5) |
| NQKFKGRETISVDKSKNTLYLQMNSLRAEDTAVYYCARVVYYSNSYWYFDVWGQGTLVTV | |
| SSASTKGPSVFPLAPSSKSTSGGTAALGCLVKDYFPEPVTVSWNSGALTSGVHTFPAVLQSSG | |
| LYSLSSVVTVPSSSLGTQTYICNVNHKPSNTKVDKKVEPKSCDKTHTCPPCPAPELLGGPSVF | |
| LFPPKPKDTLMISRTPEVTCVVVDVSHEDPEVKFNWYVDGVEVHNAKTKPREEQYNSTYRV | |
| VSVLTVLHQDWLNGKEYKCKVSNKALPAPIEKTISKAKGQPREPQVYTLPPSREEMTKNQVS | |
| LTCLVKGFYPSDIAVEWESNGQPENNYKTTPPVLDSDGSFFLYSKLTVDKSRWQQGNVFSCS | |
| VMHEALHNHYTQKSLSLSPGK. | |

In another preferred aspect, the capture reagent is a monoclonal antibody, preferably a murine antibody, and more preferably antibody 8A3 or antibody 8C5. These antibodies have the isotype IgG29. In such preferred aspect, the antibody 8A3 may be used as capture reagent and detectable antibody, or antibody 8C5 is used as capture reagent and antibody 8A3 is used as detectable antibody.

In a still preferred embodiment, the assay method comprises the steps of: (a) contacting and incubating the biological sample with the capture reagent immobilized to a solid support so as to bind any of the antibody of interest present in the sample with the capture reagent; (b) separating the biological sample from the immobilized capture reagent bound to any of the antibody of interest present; (c) contacting the immobilized capture reagent bound to any of the antibody of interest present with a detectable anti-idiotypic antibody against the antibody of interest, said detectable antibody binding to the idiotype of the antibody of interest but not to the idiotype of at least one other antibody in the sample that binds to the protein; and (d) measuring the level of any of the antibody of interest bound to the capture reagent using a detection means for the detectable antibody.

In such a method, preferably the immobilized capture reagent is coated on a microtiter plate. Also preferred is wherein the detectable antibody is directly detectable, and/or wherein the detectable antibody is amplified by a fluorimetric or colorimetric reagent. In another embodiment, the detectable antibody is biotinylated and the detection means is avidin or streptavidin-horseradish peroxidase (HRP).

In a still further aspect, the invention provides an antibody 8A3 comprising SEQ ID NOS:7 and 9 for the heavy and light chains, respectively, and obtainable from or produced by hybridoma 8A3.10 deposited under ATCC number PTA-5914.

In yet another embodiment, the invention provides an antibody 8C5 obtainable from or produced by hybridoma 8C5.1 deposited under ATCC number PTA-5915.

Both these antibodies may be conjugated to a detectable label.

In another aspect, the invention provides a hybridoma 8C5.1 or 8A3.10 deposited under ATCC deposit number PTA-5915 or PTA-5914, respectively.

In a still further embodiment, the invention provides an immunoassay kit for specifically detecting in a biological sample an antibody of interest that binds to a cell-surface, multi-transmembrane protein comprising an intervening extracellular domain of less than about 75 amino acids, the kit comprising: (a) a container containing, as a capture reagent, an anti-idiotypic antibody binding to the idiotype of the antibody of interest but not to the idiotype of at least one other antibody in the sample that binds to the protein; (b) a container containing a detectable anti-idiotypic antibody that binds to the idiotype of the antibody of interest but not to the idiotype of at least one other antibody in the sample that binds to the protein; and (c) instructions for detecting said antibody of interest.

Preferably, the kit is useful in an ELISA method for detecting the antibody of interest, more preferably a cell-based ELISA method. Also, in a preferred embodiment the kit further comprises a solid support for the capture reagent, wherein preferably the capture reagent is immobilized on the solid support such as being coated on a microtiter plate. The kit may further comprise a detection means for the detectable antibodies, such as avidin or streptavidin-HRP. The kit may further comprise purified antibody of interest as a standard. In other preferred embodiments, the capture reagent and detectable antibody are monoclonal antibodies, and they may be the same or different. The protein is preferably CD20, and the antibody of interest is preferably a humanized antibody, more preferably a humanized 2H7 antibody.

The method herein uses specific anti-idiotypic antibodies as coat and detection agents to solve the problem of specifically detecting antibodies to cell-surface proteins with small extracellular domains. It is preferably in a cell-based format, more preferably using live cells, and still more preferably live suspension WIL2 cells or live adherent transfected Chinese hamster ovary (CHO) cells. The assay can overcome interference from other antibodies to reduce non-specific sticking and background. It represents a clean, reproducible assay for antibodies in biological samples, especially serum, giving a high throughput so that many samples can be run at once, as through an ELISA that is automated using one plate.

### BRIEF DESCRIPTION OF THE FIGURES

FIGS. 1A and 1B show titration curves of chimeric anti-CD20 IgG (solid line) and humanized anti-CD20 IgG (dashed line) in suspension WIL2 binding assay (FIG. 1A) and adherent 2H3 CHO clone binding assay (FIG. 1B). The background readings (OD 450 nm) were 0.064±003 and 0.116±0.003 for the WIL2 and CHO binding assays, respectively. The relative activities of humanized anti-CD20 IgG were calculated to be 0.68±0.04 and 0.70±0.02 for the WIL2 and 2H3 binding assays, respectively (n=2).

FIG. 2 shows titration curves of RITUXAN® binding to CHO clones (Table 1) with differing CD20 expression. Clone 3G8, which had little CD20 expression (mean fluorescence of 0.5 compared to 9.8 for clone 4H10), was also included for comparison. The assay was performed using 300,000 cells/well in the suspension format. The background reading (OD 450 nm) for clone 4H10 was 0.016±0.001 (n=2).

FIGS. 3A and 3B show specificity of anti-idiotypic antibodies 8C5 (FIG. 3A) and 8A3 (FIG. 3B). Serially diluted humanized anti-CD20 IgG, HERCEPTIN® (Carter et al., Proc. Natl. Acad. Sci. USA, 89: 4285-4289 (1992)), anti-vascular endothelial growth factor (VEGF) (Presta et al., Cancer Res., 57: 4593-4599 (1997)), E25 (Presta et al., J. Immunology, 151: 2623-2632 (1993)), RITUXAN®, and normal human IgG (Zymed, South San Francisco, Calif.) were incubated on 8C5- or 8A3-coated ELISA plates and bound antibody was detected using goat anti-human IgG Fc-HRP. The background reading (OD 450 nm) was 0.012±0.001 (n=2).

FIGS. 4A and 4B show an ELISA using anti-idiotypic antibody 8C5 for coat and biotinylated 8A3 for detection. FIG. 4A shows titration curves of humanized anti-CD20 IgG in buffer (solid line) or 20% human serum (dashed line). The background readings (OD 450 nm) were 0.020±0.004 and 0.015±0.003 in buffer or 20% human serum, respectively (n=3). FIG. 4B shows titration curves of parent murine anti-CD20 in buffer (solid line) or in 10% mouse serum (dashed line). The background readings (OD 450 nm) were 0.057±0.004 and 0.018±0.001 in buffer or 10% mouse serum, respectively (n=2).

FIGS. 5A-5E show the amino acid and nucleotide sequences of antibody 8A3, with FIG. 5A showing the amino acid sequence of the heavy chain of MAb 8A3 (SEQ ID NO:6), FIG. 5B showing the amino acid sequence of the heavy chain of MAb 8A3 without the 23-amino-acid signal sequence (SEQ ID NO:7), FIG. 5C showing the amino acid sequence of the light chain of MAb 8A3 (SEQ ID N O :8), FIG. 5D showing the amino acid sequence of the light chain of MAb 8A3 without the 23-amino-acid signal sequence (SEQ ID NO:9), and FIG. 5E showing the nucleotide sequence encoding the light and heavy chains of MAb 8A3, wherein nucleotide residue 40 is the beginning of the signal sequence for the light chain (SEQ ID NO: 10).

FIG. 6A is a sequence alignment comparing the amino acid sequences of the light-chain variable domain (V_{L}) of each of murine 2H7 (SEQ ID NO: 11), humanized 2H7.v16 variant (SEQ ID NO: 12), and the human kappa light-chain subgroup I (SEQ ID NO: 13). The CDRs of V_{L} of 2H7 and hu2H7.v16 are as follows: CDR1 (SEQ ID NO:14), CDR2 (SEQ ID NO:15), and CDR3 (SEQ ID NO: 16).

FIG. 6B is a sequence alignment comparing the amino acid sequences of the heavy-chain variable domain (V_{H}) of each of murine 2H7 (SEQ ID NO: 17), humanized 2H7.v16 variant (SEQ ID NO: 18), and the human consensus sequence of the heavy-chain subgroup m (SEQ ID NO: 19). The CDRs of V_{H} of 2H7 and hu2H7. v16 are as follows: CDR1 (SEQ ID NO:20), CDR2 (SEQ ID NO:21), and CDR3 (SEQ ID NO:22).

In FIG. 6A and FIG. 6B, the CDR1, CDR2, and CDR3 in each chain are enclosed within brackets, flanked by the framework regions, FR1-FR4, as indicated. 2H7 refers to the murine 2H7 antibody. The asterisks in between two rows of sequences indicate the positions that are different between the two sequences. Residue numbering is according to Kabat et al., Sequences of Immunological Interest, 5th Ed. Public Health Service, National Institutes of Health, Bethesda, Md. (1991), with insertions shown as a, b, c, d, and e.

FIGS. 7A and 7B show the amino acid sequences of the 2H7.v16 L chain, with FIG. 7A showing the complete L chain containing the first 19 amino acids before DIQ that are the secretory signal sequence not present in the mature polypeptide chain (SEQ ID NO:23), and FIG. 7B showing the mature polypeptide L chain (SEQ ID NO:24).

FIGS. 8A and 8B show the amino acid sequences of the 2H7.v16 H chain, with FIG. 8A showing the complete H chain containing the first 19 amino acids before EVQ that are the secretory signal sequence not present in the mature polypeptide chain (SEQ ID NO:25), and FIG. 8B showing the mature polypeptide H chain (SEQ ID NO:26). Aligning the V_{H} sequence in FIG. 6B (SEQ ID NO: 18) with the complete H-chain sequence, the human γ1 constant region is from amino acid position 114-471 in SEQ ID NO:25.

FIGS. 9A and 9B show the amino acid sequences of the 2H7.v31 H chain, with FIG. 9A showing the complete H chain containing the first 19 amino acids before EVQ that are the secretory signal sequence not present in the mature polypeptide chain (SEQ ID NO:27), and FIG. 9B showing the mature polypeptide H chain (SEQ ID NO:28). The L chain is the same as for 2H7.v16 (see FIG. 7).

FIG. 10 is a sequence alignment comparing the light-chain amino acid sequences of the humanized 2H7.v16 variant (SEQ ID NO: 12) and humanized 2H7.v138 variant (SEQ ID NO:33).

FIG. 11 is a sequence alignment comparing the heavy-chain amino acid sequences of the humanized 2H7.v16 variant (SEQ ID NO: 18) and humanized 2H7.v138 variant (SEQ ID NO:34).

FIG. 12 is a sequence alignment comparing the light-chain amino acid sequences of the humanized 2H7.v16 variant (SEQ ID NO: 18) and humanized 2H7.v511 (SEQ ID NO: ), wherein residues are numbered throughout using the EU numbering system. With respect to the EU antibody, v16 and v511 have a deletion at position 30 in the variable domain; therefore, S30 in the sequential numbering of v16/v511 is assigned as position 31 (EU).

FIG. 13 is a sequence alignment comparing the heavy-chain amino acid sequences of the humanized 2H7.v16 variant (SEQ ID NO: 18) and humanized 2H7.v511 (SEQ ID NO: ), wherein residues are numbered throughout using the EU numbering system. In the variable domain, v16 and v511 have an insertion of five residues, designated 104a-e, compared to the EU antibody. The first constant domain, CH1, begins at position 118 (EU).

FIG. 14 is a sequence alignment comparing the light-chain amino acid sequences of the humanized 2H7.v16 variant (SEQ ID NO: 18) and humanized 2H7.v511 (SEQ ID NO:38), wherein residues are numbered using the Kabat numbering system. Note that v16 and v511 have a deletion at Kabat position 31; therefore residue Y31 in sequential numbering is designated as Y32 (Kabat).

FIG. 15 is a sequence alignment comparing the heavy-chain amino acid sequences of the humanized 2H7.v16 variant (SEQ ID NO:18) and humanized 2H7.v511 (SEQ ID NO:39), wherein residues in the variable domain (1-113) are numbered using the Kabat numbering system. Residues in the constant domains (118-447) are numbered using the EU numbering system.

FIG. 16 shows the standard curves of three mouse 2H7 variants in the anti-idiotypic-antibody-based ELISA herein (v16, v96, and v327) in mouse serum using MAb 85C as coat antibody and biotinylated MAb 8A3 (8A3-bio) as detection antibody.

FIG. 17 shows the standard curves of four humanized 2H7 variants in the anti-idiotypic-antibody-based ELISA herein (v16, v114, v488, and v511) in mouse serum using MAb 8C5 as coat antibody and biotinylated MAb 8A3 (8A3-bio) as detection antibody.

### DETAILED DESCRIPTION OF PREFERRED EMBODIMENTS

### I. Definitions

The term “cell-surface, multi-transmembrane protein comprising an intervening extracellular domain (ECD) of less than about 75 amino acids” refers to a protein that has domain(s) that cross the membrane and only a short extracellular domain that can be used for generating antibodies. By “short” is meant generally a range of about 20 to less than about 75 amino acids, more preferably about 20 to about 50 amino acids. The multi-transmembrane refers to more than two transmembrane domains in the protein. Examples of such proteins include, but are not limited to, G-protein coupled receptors such as receptors for the hormone relaxin (e.g., LGR7 and LGR8) and chemokine receptors, and B-cell surface markers that meet the above definition of the protein, such as the CD20 antigen (CD20).

The term “chemokine” refers to all known chemotactic cytokines expressed within mammalian organisms that mediate the recruitment and infiltration of leukocytes into tissues. The term “chemokine” includes but is not limited to all mammalian members of the C, CC, CXC, and CXXXC families of chemotactic cytokines, classified within the art based upon the distribution of cystine residues therein. The phrase “chemokine receptor” refers to transmembrane proteins, exemplified in the art, that interact with one or more chemokines. The category of “chemokine receptor” includes, but is not limited to, all chemokine receptors classified within the art as CR, CCR, CXCR, and CXXXCR. The term “cytokine” refers to all human cytokines known within the art that bind extracellular receptors upon the cell surface and thereby modulate cell function, including but not limited to IL-2, IFN-gamma, TNF-alpha, IL-4, IL-5, IL-6, IL-9, IL-10, and IL-13. Examples of chemokine receptors include those receptors for interleukin-8 (IL-8), RANTES (regulated upon activation, normal T-cell expressed, and presumably secreted), macrophage inflammatory protein-1 (MIP-1), CCR1, CCR2, CCR2B, CCR3, CCR4, CCR5, CCR6, CCR7, CCR8, CCR9, CCR10, CCR 11, CXCR1, CXCR2, CXCR3, CXCR4, CXCR5, CXCR6, CX3CR1, XCR1, the orphan chemokine receptor GPR-9-6, platelet factor 4 (PF4), monocytes, chemotactic and activating factor (MCAF), and neutrophil-activating protein-2 (NAP-2), which have small intervening ECDs.

A “B-cell surface marker” or “B-cell surface antigen” herein is an antigen expressed on the surface of a B cell that can be targeted with an antagonist that binds thereto and also meets the criteria above for the multi-transmembrane protein. Exemplary B-cell surface markers include the CD10, CD19, CD20, CD21, CD23, CD24, CD37, CD40, CD53, CD72, CD73, CD74, CDw75, CDw76, CD77, CDw78, CD79a, CD79b, CD80, CD81, CD82, CD83, CDw84, CD85, and CD86 leukocyte surface markers. (For descriptions, see The Leukocyte Antigen Facts Book, 2nd Edition, Barclay et al., ed. (Academic Press, Harcourt Brace & Co., New York: 1997).) Other B-cell surface markers include RP105, FcRH2, CD79A, C79B, B cellCR2, CCR6, CD72, P2X5, HLA-DOB, CXCR5, FCER2, BR3, Btig, NAG14, SLGC16270, FcRH1, IRTA2, ATWD578, FcRH3, IRTA1, FcRH6, BCMA, and 239287_at. The B-cell surface marker of particular interest is preferentially expressed on B cells compared to other non-B-cell tissues of a mammal and may be expressed on both precursor B cells and mature B cells.

The “CD20” antigen, or “CD20,” is an approximately 35-kDa, non-glycosylated phosphoprotein found on the surface of greater than 90% of B cells from peripheral blood or lymphoid organs. CD20 is present on both normal B cells as well as malignant B cells, but is not expressed on stem cells. Other names for CD20 in the literature include “B-lymphocyte-restricted antigen” and “Bp35”. The CD20 antigen is described in Clark et al., PNAS (USA), 82:1766 (1985), for example.

“Mammal” for purposes of treatment refers to any animal classified as a mammal, including humans, domestic, and farm animals, and zoo, sports, or pet animals, such as dogs, horses, cats, sheep, pigs, cows, etc. Preferably, the mammal is human.

The terms “cancer”, “cancerous”, and “malignant” refer to or describe the physiological condition in mammals that is typically characterized by unregulated cell growth. Examples of cancer include, but are not limited to, carcinoma including adenocarcinoma, lymphoma, blastoma, melanoma, sarcoma, and leukemia. More particular examples of such cancers include squamous cell cancer, small-cell lung cancer, non-small cell lung cancer, gastrointestinal cancer, Hodgkin's and non-Hodgkin's lymphoma, pancreatic cancer, glioblastoma, cervical cancer, ovarian cancer, liver cancer such as hepatic carcinoma and hepatoma, bladder cancer, breast cancer, colon cancer, colorectal cancer, endometrial carcinoma, salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer. The preferred cancers for treatment herein are breast, colon, lung, colorectal, prostate, lymphoma such as non-Hodgkin's lymphoma, and melanoma.

The term “chemokine-mediated disease” refers to a disease that can be treated or prevented or its symptoms ameliorated by an antagonist to a chemokine receptor. Such diseases include, for example, psoriasis, atopic dermatitis, asthma, chronic obstructive pulmonary disorder, adult respiratory disease, arthritis, inflammatory bowel disease, Crohn's disease, ulcerative colitis, septic shock, endotoxic shock, gram-negative sepsis, toxic shock syndrome, stroke, cardiac and renal reperfusion injury, glomerulonephritis, thrombosis, Alzheimer's disease, graft-versus-host reaction, allograft rejection, malaria, acute respiratory distress syndrome, delayed-type hypersensitivity reaction, atherosclerosis, cerebral and cardiac ischemia, osteoarthritis, multiple sclerosis, restenosis, angiogenesis, osteoporosis, gingivitis, respiratory viruses, herpes viruses, hepatitis viruses, HIV, Kaposi's sarcoma-associated virus, meningitis, cystic fibrosis, pre-term labor, cough, pruritis, multi-organ dysfunction, trauma, strains, sprains, contusions, psoriatic arthritis, herpes, encephalitis, CNS vasculitis, traumatic brain injury, CNS tumors, subarachnoid hemorrhage, post surgical trauma, interstitial pneumonitis, hypersensitivity, crystal induced arthritis, acute and chronic pancreatitis, acute alcoholic hepatitis, necrotizing enterocolitis, chronic sinusitis, angiogenic ocular disease, ocular inflammation, retinopathy of prematurity, diabetic retinopathy, wet-type macular degeneration, corneal neovascularization, polymyositis, vasculitis, acne, gastric and duodenal ulcer, celiac disease, esophagitis, glossitis, airflow obstruction, airway hyperresponsiveness, bronchiectasis, bronchiolitis, bronchiolitis obliterans, chronic bronchitis, cor pulmonae, dyspnea, emphysema, hypercapnea, hyperinflation, hypoxemia, hyperoxia-induced inflammation, hypoxia, surgical lung volume reduction, pulmonary fibrosis, pulmonary hypertension, right ventricular hypertrophy, peritonitis associated with continuous ambulatory peritoneal dialysis, granulocytic ehrlichiosis, sarcoidosis, small-airway disease, ventilation-perfusion mismatching, wheeze, colds, gout, alcoholic liver disease, lupus, burn therapy, periodontitis, transplant reperfusion injury, early transplantation, rheumatoid arthritis (all types), and cancer. The inflammatory bowel diseases include acute and chronic inflammatory bowel disease, and HIV includes AIDS. Exemplary drugs that can be used in conjunction with an antibody against a chemokine receptor to treat such disease include those disclosed in U.S. Pub. No. US 20040053953.

The term “detecting” is used in the broadest sense to include both qualitative and quantitative measurements of a target molecule. In one aspect, the detecting method as described herein is used to identify the mere presence of the antibody of interest in a biological sample. In another aspect, the method is used to test whether the antibody of interest in a sample is present at a detectable level. In yet another aspect, the method can be used to quantify the amount of the antibody of interest in a sample and further to compare the antibody levels from different samples.

The term “antibody of interest” refers to an antibody that binds to a protein as described herein. Such an antibody is preferably a monoclonal antibody, more preferably a rodent, e.g., murine antibody or a humanized antibody, still more preferably a humanized antibody. Examples of such antibodies include an antibody or functional fragment thereof that binds to a mammalian CC-chemokine receptor (CCR), such as C-chemokine receptor 2 (also referred to as CCR2, CKR-2, MCP-1RA, or MCP-1RB) or portion of the receptor (e.g., anti-CCR2). Such antibody, for example, may have specificity for human or rhesus CCR2 or a portion thereof and/or block binding of a ligand (e.g., MCP-1, MCP-2, MCP-3, or MCP-4) to the receptor and inhibit function associated with binding of the ligand to the receptor (e.g., leukocyte trafficking). Such antibody is preferably murine monoclonal antibody (MAb) LS132.1D9 (1D9) or an antibody that can compete with 1D9 for binding to human CCR2 or a portion of human CCR2, such as the humanized antibodies as described in U.S. Pat. No. 6,696,550. Examples also include antibodies that bind to chemokine receptors CCR3 or CCR10, the preparation of which is described in U.S. Pat. No. 6,692,922. Another example is an antibody to chemokine receptor GPR-9-6, such as MAb 3C3, which selectively reacts with GPR-9-6 transfectants (see U.S. Pat. No. 6,689,570). Further examples are antibodies that specifically bind and/or modulate one topology of a chemokine receptor, but not a second topology of the receptor, as described, for example, in U.S. Pub. No. US 20040018563. Another example is isolated heterogeneous anti-leukocyte receptor IgM antibodies that target at least CCR5, CCR3, CXCR4, and/or CCR2B, as described in U.S. Pat. No. 6,610,834. Further examples are the fully human anti-CD3 antibodies such as fhCD3mAb disclosed in U.S. Pub. No. US 20030216551 that interfere with the in vivo role of mammalian chemokine receptors when administered in vivo. Additional examples are monoclonal human antibodies against human CXCR4 capable of inhibiting HIV infection and chemotaxis in human breast cancer cells, such as antibodies binding to loop 6 of human CXCR4, e.g., Ab124 and Ab125, as described in U.S. Pat. Pub. US 20030206909. The most preferred antibody of interest herein is a humanized 2H7 antibody.

The term “biological sample” refers to any biological substance that may contain the antibody of interest. A sample can be biological fluid, such as whole blood or whole blood components including red blood cells, white blood cells, platelets, serum and plasma, ascites, urine, vitreous fluid, lymph fluid, synovial fluid, follicular fluid, seminal fluid, amniotic fluid, milk, saliva, sputum, tears, perspiration, mucus, cerebrospinal fluid, and other constituents of the body that may contain the analyte of interest, as well as tissue culture medium and tissue extracts such as homogenized tissue, and cellular extracts. Preferably, the sample is a body sample from any animal, but preferably is from a mammal, more preferably from a human subject, for example, when measuring an antibody such as a humanized antibody in a clinical sample, or a mouse subject, for example, when measuring the parent mouse antibody in mouse samples, particularly the serum. Most preferably, such biological sample is from clinical patients. The preferred biological sample herein is serum, plasma or urine, more preferably serum, and most preferably serum from a clinical patient.

The term “capture reagent” or “coat antibody” refers to an anti-idiotypic antibody or mixture of such antibodies that bind an idiotype of the antibody of interest and are capable of binding and capturing the antibody of interest in a biological sample such that under suitable conditions, the complex of capture reagent and antibody of interest can be separated from the rest of the sample. Anti-idiotypic antibodies are antibodies that bind to the V_{H} and/or V_{L} domain of the cognate antibody, in this case the antibody of interest. Typically, such anti-idiotypic antibodies are prepared by immunizing a mammal such as a mouse with the antibody of interest and producing a hybridoma and selecting from the panel of antibodies derived from the hybridoma those antibodies that give the cleanest signal in the assay, whether for the capture reagent or the detectable antibody. Typically, the capture reagent is immobilized or immobilizable. Preferably, such anti-idiotypic antibodies are monoclonal antibodies, more preferably rodent antibodies, still more preferably murine or rat antibodies, and most preferably murine antibodies.

The term “detectable antibody” refers to an anti-idiotypic antibody or mixture of such antibodies that bind an idiotype of the antibody of interest and are capable of being detected either directly through a label amplified by a detection means, or indirectly through, e.g., another antibody that is labeled. For direct labeling, the antibody is typically conjugated to a moiety that is detectable by some means. The preferred detectable antibody is biotinylated antibody. The preferred such anti-idiotypic antibodies are monoclonal antibodies, more preferably rodent antibodies, still more preferably murine or rat antibodies, and most preferably murine antibodies.

The term “detection means” refers to a moiety or technique used to detect the presence of the detectable antibody through signal reporting that is then read out in the assay herein. It includes reagents that amplify the immobilized label such as the label captured onto a microtiter plate. Preferably, the detection means is avidin or streptavidin-HRP.

The term “antibody” herein is used in the broadest sense and specifically covers intact monoclonal antibodies, polyclonal antibodies, multispecific antibodies (e.g. bispecific antibodies) formed from at least two intact antibodies, and antibody fragments so long as they exhibit the desired biological activity.

“Antibody fragments” comprise a portion of an intact antibody, preferably comprising the antigen-binding or variable region thereof. Examples of antibody fragments include Fab, Fab′, F(ab′)₂, and Fv fragments; diabodies; linear antibodies; single-chain antibody molecules; and multispecific antibodies formed from antibody fragments.

For the purposes herein, an “intact antibody” is one comprising heavy- and light-chain variable domains as well as an Fc region.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light-chain and heavy-chain variable domains.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al. Nature 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable-domain antigen-binding sequences derived from a non-human primate (e.g. Old World Monkey, such as baboon, rhesus or cynomolgus monkey) and human constant-region sequences (U.S. Pat No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made further to refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature, 321:522-525 (1986); Riechmann et al., Nature, 332:323-329 (1988); and Presta, Curr. Op. Struct. Biol., 2:593-596 (1992).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions in both the light-chain and the heavy-chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody-dependent cellular cytotoxicity (ADCC).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment that contains a complete antigen-recognition and antigen-binding site. This region consists of a dimer of one heavy-chain and one light-chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy-chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains that enables the scFv to form the desired structure for antigen binding. For a review of scFv, see Plüickthun in The Pharmacology of Monoclonal Antibodies, vol. 113, Rosenburg and Moore eds., Springer-Verlag, N. Y., pp. 269-315 (1994).

The term “hypervariable region” when used herein refers to the amino acid residues of an antibody that are responsible for antigen binding. The hypervariable region comprises amino acid residues from a “complementarity-determining region” or “CDR”(e.g. residues 24-34 (L1), 50-56 (L2) and 89-97 (L3) in the light-chain variable domain and 31-35 (H1), 50-65 (H2) and 95-102 (H3) in the heavy-chain variable domain; Kabat et al, Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk J. Mol. Biol. 196:901-917 (1987)). “Framework” or “FR” residues are those variable domain residues other than the hypervariable region residues as herein defined.

Examples of antibodies that bind the CD20 antigen include: “C2B 8” which is now called “rituximab” (“RITUXAN®”) (U.S. Pat. No. 5,736,137); the yttrium-[90]-labeled 2B8 murine antibody designated “Y2B8” or “Ibritumomab Tiuxetan” ZEVALIN® (U.S. Pat. No. 5,736,137); murine IgG2a “B 1, ” also called “Tositumomab,” optionally labeled with ¹³¹I to generate the “¹³¹I-B1” antibody (iodine I131 tositumomab, BEXXAR™) (U.S. Pat. No. 5,595,721); murine monoclonal antibody “1F5”(Press et al., Blood, 69(2):584-591 (1987) and “framework patched” or humanized 1F5 (WO03/002607, Leung, S.); ATCC deposit HB-96450); murine 2H7 and chimeric 2H7 antibody (U.S. Pat. No. 5,677,180); a humanized 2H7; huMax-CD20 (Genmab, Denmark); AME-133 (Applied Molecular Evolution); and monoclonal antibodies L27, G28-2, 93-1B3, B-C1 or NU-B2 available from the International Leukocyte Typing Workshop (Valentine et al., In: Leukocyte Typing III (McMichael, Ed., p. 440, Oxford University Press (1987)).

The term “rituximab” or “RITUXAN®” herein refers to the genetically engineered chimeric murine/human monoclonal antibody directed against the CD20 antigen and designated “C2B8” in U.S. Pat. No. 5,736,137, including fragments thereof that retain the ability to bind CD20.

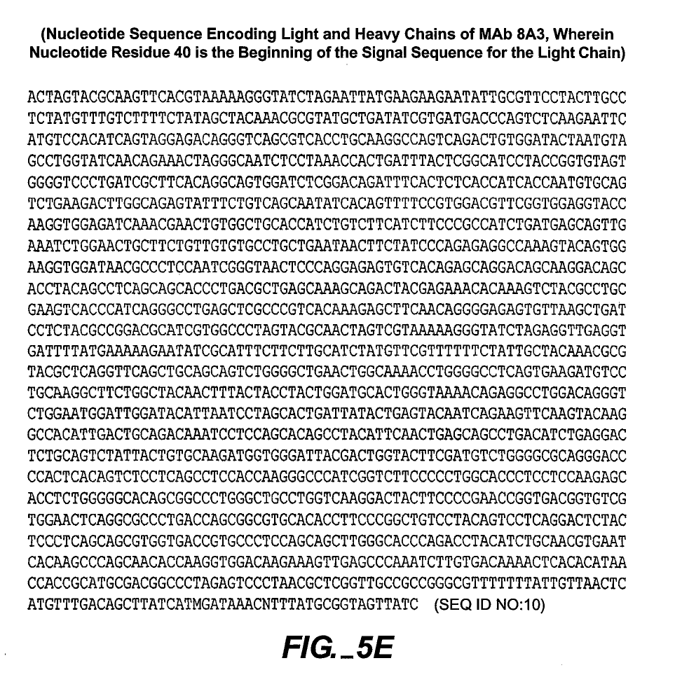

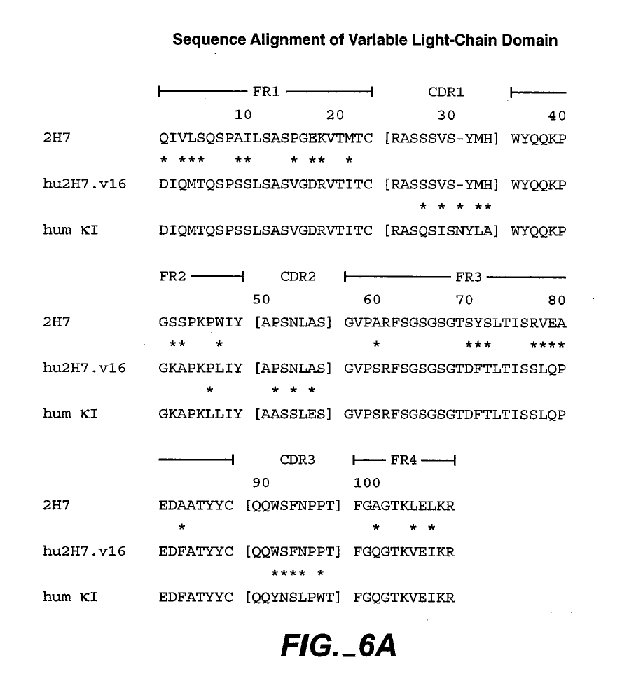

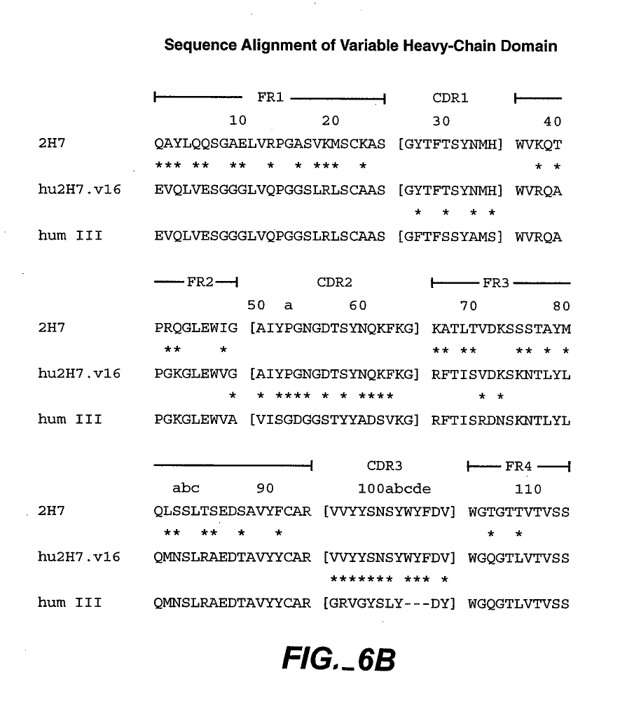

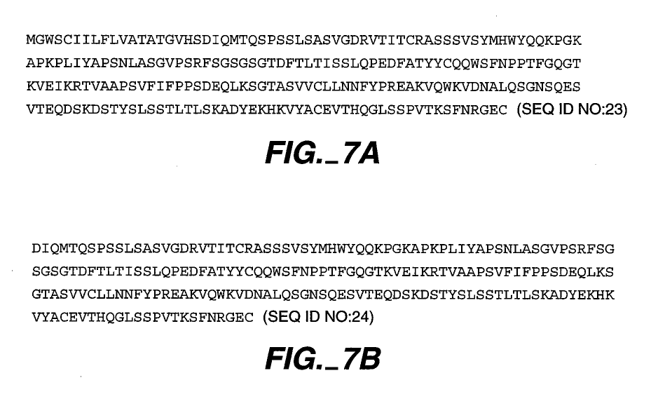

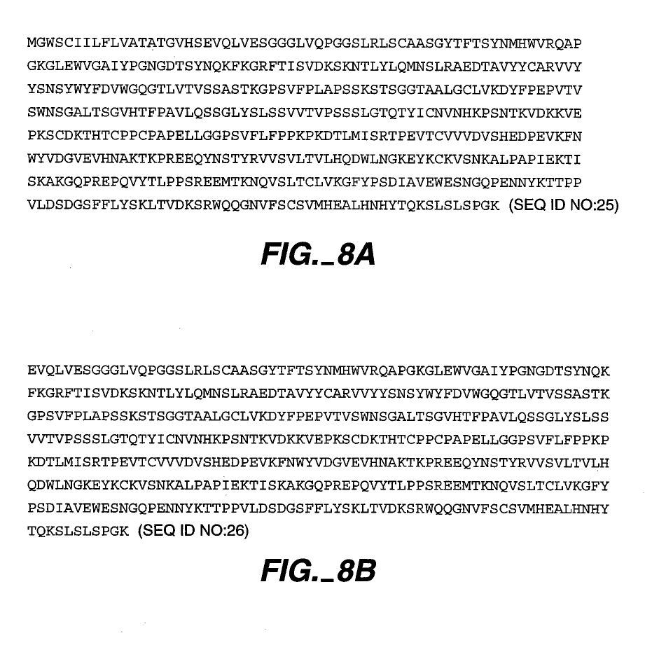

Purely for the purposes herein, “humanized 2H7” refers to a humanized antibody that binds human CD20, or an antigen-binding fragment thereof, wherein the antibody is effective to deplete primate B cells in vivo, the antibody comprising in the H-chain variable region (V_{H}) at least a CDR3 sequence of SEQ ID NO:22 (FIG. 6B) from an anti-human CD20 antibody and substantially the human consensus framework (FR) residues of the human heavy-chain subgroup III (V_{H}III). In a preferred embodiment, this antibody further comprises the H-chain CDR1 sequence of SEQ ID NO:20 and CDR2 sequence of SEQ ID NO:21, and more preferably further comprises the L-chain CDR1 sequence of SEQ ID NO: 14, CDR2 sequence of SEQ ID NO: 15, CDR3 sequence of SEQ ID NO: 16 and substantially the human consensus framework (FR) residues of the human light-chain κ subgroup I (VκI), wherein the V_{H} region may be joined to a human IgG chain constant region, wherein the region may be, for example, IgG1 or IgG3. In a preferred embodiment, such antibody comprises the V_{H} sequence of SEQ ID NO:18 (v16, as shown in FIG. 6B), optionally also comprising the V_{L} sequence of SEQ ID NO:12 (v16, as shown in FIG. 6A), which may have the amino acid substitutions of D56A and N100A in the H chain and S92A in the L chain (v.96). A more preferred such antibody is 2H7.v16 having the light- and heavy-chain amino acid sequences of SEQ ID NOS:26 and 28, respectively, as shown in FIGS. 7B and 8B. Another preferred embodiment is where the antibody is 2H7.v31 having the light- and heavy-chain amino acid sequences of SEQ ID NOS:26 and 30, respectively, as shown in FIGS. 7B and 9B. The antibody herein may further comprise at least one amino acid substitution in the Fc region that improves ADCC and/or CDC activity, such as one wherein the amino acid substitutions are S298A/E333A/K334A, more preferably 2H7.v31 having the heavy-chain amino acid sequence of SEQ ID NO:28 (as shown in FIG. 9B). Any of these antibodies may further comprise at least one amino acid substitution in the Fc region that decreases CDC activity, for example, comprising at least the substitution K322A. Such antibodies preferably are 2H7.v114 or 2H7.v115 having at least 10-fold improved ADCC activity as compared to RITUXAN®.

A preferred humanized 2H7 is an intact antibody or antibody fragment comprising the variable light-chain sequence:

| | |
|---|---|
| DIQMTQSPSSLSASVGDRVTITCRASSSVSYMHWYQQKPGKAPKPLIYAPSNLASGVPSRESG | (SEQ ID NO: 1) |
| SGSGTDFTLTISSLQPEDFATYYCQQWSFNPPTFGQGTKVEIKR; | |

and the variable heavy-chain sequence:

| | |
|---|---|
| EVQLVESGGGLVQPGGSLRLSCAASGYTFTSYNMHWVRQAPGKGLEWVGAIYPGNGDTSY | (SEQ ID NO: 2) |
| NQKEKGRFTISVDKSKNTLYLQMNSLRAEDTAVYYCARVVYYSNSYWYFDVWGQGTLVTV | |
| SS | |

Where the humanized 2H7 antibody is an intact antibody, preferably it comprises the light-chain amino acid sequence:

| | |
|---|---|
| DIQMTQSPSSLSASVGDRVTITCRASSSVSYMHWYQQKPGKAPKPLIYAPSNLASGVPSRFSG | (SEQ ID NO: 3) |
| SGSGTDFTLTISSLQPEDFATYYCQQWSFNPPTFGQGTKVEIKRTVAAPSVFIFPPSDEQLKSG | |
| TASVVCLLNNEYPREAKVQWKVDNALQSGNSQESVTEQDSKDSTYSLSSTLTLSKADYEKH | |
| KVYACEVTHQGLSSPVTKSFNRGEC; | |

and the heavy-chain amino acid sequence:

| | |
|---|---|
| EVQLVESGGGLVQPGGSLRLSCAASGYTFTSYNMHWVRQAPGKGLEWVGAIYPGNGDTSY | (SEQ ID NO: 4) |
| NQKFKGRFTISVDKSKNTLYLQMNSLRAEDTAVYYCARVVYYSNSYWYFDVWGQGTLVTV | |
| SSASTKGPSVFPLAPSSKSTSGGTAALGCLVKDYFPEPVTVSWNSGALTSGVHTFPAVLQSSG | |
| LYSLSSVVTVPSSSLGTQTYICNVNHKPSNTKVDKKVEPKSCDKTHTCPPCPAPELLGGPSVF | |
| LFPPKPKDTLMISRTPEVTCVVVDVSHEDPEVKFNWYVDGVEVHNAKTKPREEQYNSTYRV | |
| VSVLTVLHQDWLNGKEYKCKVSNKALPAPIEKTISKAKGQPREPQVYTLPPSREEMTKNQVS | |
| LTCLVKGFYPSDIAVEWESNGQPENNYKTTPPVLDSDGSFFLYSKLTVDKSRWQQGNVFSCS | |
| VMHEALHNHYTQKSLSLSPGK | |

or the heavy-chain amino acid sequence:

| | |
|---|---|
| EVQLVESGGGLVQPGGSLRLSCAASGYTFTSYNMHWVRQAPGKGLEWVGAIYPGNGDTSY | (SEQ ID NO: 5) |
| NQKFKGRFTISVDKSKNTLYLQMNSLRAEDTAVYYCARVVYYSNSYWYFDVWGQGTLVTV | |
| SSASTKGPSVFPLAPSSKSTSGGTAALGCLVKDYFPEPVTVSWNSGALTSGVHTFPAVLQSSG | |
| LYSLSSVVTVPSSSLGTQTYICNVNHKPSNTKVDKKVEPKSCDKTHTCPPCPAPELLGGPSVF | |
| LFPPKPKDTLMISRTPEVTCVVVDVSHEDPEVKFNWYVDGVEVHNAKTKPREEQYNSTYRV | |
| VSVLTVLHQDWLNGKEYKCKVSNKALPAPIEKTISKAKGQPREPQVYTLPPSREEMTKNQVS | |
| LTCLVKGFYPSDIAVEWESNGQPENNYKTTPPVLDSDGSFFLYSKLTVDKSRWQQGNVFSCS | |
| VMHEALHNHYTQKSLSLSPGK. | |

The term “instructions” is used to refer to instructions customarily included in commercial packages of therapeutic products that contain information about the indications, usage, dosage, administration, contraindications and/or warnings concerning the use of such therapeutic products.

### II. MODES FOR CARRYING OUT THE INVENTION

The assay described herein is an ELISA that utilizes anti-idiotypic antibodies as capture reagents and detectable antibodies for an antibody of interest. Preferably, the ELISA is cell-based. In the first step of the assay the biological sample suspected of containing or containing the antibody of interest is contacted and incubated with the capture (or coat) antibodies so that the capture antibodies capture or bind to the antibody of interest so that it can be detected in a detection step. The detection step involves use of the detectable anti-idiotypic antibody, which, when contacted with any of the bound antibody of interest, binds to the antibody of interest, if present, and a detection means is used to detect the label on the antibody and hence the presence or amount of antibody of interest present.

In a more preferred embodiment, the assay utilizes the following steps.

First Step

In the first step of the assay herein, the biological sample suspected of containing or containing the antibody of interest as defined herein is contacted and incubated with the immobilized capture (or coat) reagents, which are anti-idiotypic antibodies directed against the antibody of interest. These antibodies are preferably monoclonal antibodies, and may be from any species, but preferably they are rodent, more preferably murine or rat, still more preferably murine, and most preferably MAb 8A3 or 8C5 derived from the hybridomas identified herein. MAb 8A3 comprises SEQ ID NOS:7 and 9 for the heavy and light chains, respectively. Hence, in a specific preferred embodiment, the immobilized anti-idiotypic antibody is a murine monoclonal antibody, most preferably MAb 8C5 or 8A3. Immobilization conventionally is accomplished by insolubilizing the capture reagents either before the assay procedure, as by adsorption to a water-insoluble matrix or surface (U.S. Pat. No. 3,720,760) or non-covalent or covalent coupling (for example, using glutaraldehyde or carbodiimide cross-linking, with or without prior activation of the support with, e.g., nitric acid and a reducing agent as described in U.S. Pat. No. 3,645,852 or in Rotmans et al., J. Immunol. Methods, 57:87-98 (1983)), or afterward, e.g., by immunoprecipitation.

The solid phase used for immobilization may be any inert support or carrier that is essentially water insoluble and useful in immunometric assays, including supports in the form of, e.g., surfaces, particles, porous matrices, etc. Examples of commonly used supports include small sheets, SEPHADEX® gels, polyvinyl chloride, plastic beads, and assay plates or test tubes manufactured from polyethylene, polypropylene, polystyrene, and the like, including 96-well microtiter plates, as well as particulate materials such as filter paper, agarose, cross-linked dextran, and other polysaccharides. Alternatively, reactive water-insoluble matrices such as cyanogens-bromide-activated carbohydrates and the reactive substrates described in U.S. Pat. Nos. 3,969,287; 3,691,016; 4,195,128; 4,247,642; 4,229,537; and 4,330,440 are suitably employed for capture-reagent immobilization. In a preferred embodiment, the immobilized capture reagents are coated on a microtiter plate, and in particular the preferred solid phase used is a multi-well microtiter plate that can be used to analyze several samples at one time. The most preferred is a MICROTEST™ or MAXISORP™ 96-well ELISA plate such as that sold as NUNC MAXISORB™ or IMMULON™.

The solid phase is coated with the capture reagents as defined above, which may be linked by a non-covalent or covalent interaction or physical linkage as desired. Techniques for attachment include those described in U.S. Pat. No. 4,376,110 and the references cited therein. If covalent, the plate or other solid phase is incubated with a cross-linking agent together with the capture reagent under conditions well known in the art such as for one hour at room temperature.

Commonly used cross-linking agents for attaching the capture reagents to the solid-phase substrate include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-((p-azidophenyl)-dithio)propioimidate yield photoactivatable intermediates capable of forming cross-links in the presence of light.

If 96-well plates are utilized, they are preferably coated with the mixture of capture reagents typically diluted in a buffer such as 0.05 M sodium carbonate by incubation for at least about 10 hours, more preferably at least overnight, at temperatures of about 4-20° C., more preferably about 4-8° C., and at a pH of about 8-12, more preferably about 9-10, and most preferably about 9.6. If shorter coating times (1-2 hours) are desired, one can use 96-well plates with nitrocellulose filter bottoms (Millipore MULTISCREEN™) or coat at 37° C. The plates may be stacked and coated long in advance of the assay itself, and then the assay can be carried out simultaneously on several samples in a manual, semi-automatic, or automatic fashion, such as by using robotics.

The coated plates are then typically treated with a blocking agent that binds non-specifically to and saturates the binding sites to prevent unwanted binding of the free ligand to the excess sites on the wells of the plate. Examples of appropriate blocking agents for this purpose include, e.g., gelatin, bovine serum albumin, egg albumin, casein, and non-fat milk. The blocking treatment typically takes place under conditions of ambient temperatures for about 1-4 hours, preferably about 1.5 to 3 hours.

After coating and blocking, the standard (purified antibody of interest) or the biological sample to be analyzed, appropriately diluted, is added to the immobilized phase. The preferred dilution rate is about 5-15%, preferably about 10%, by volume. Buffers that may be used for dilution for this purpose include (a) phosphate-buffered saline (PBS) containing 0.5% BSA, 0.05% TWEEN 20™ detergent (P20), 0.05% PROCLIN™ 300 antibiotic, 5 mM EDTA, 0.25% 3-((3-cholamidopropyl)dimethylammonio)-1-propanesulphonate (CHAPS) surfactant, 0.2% beta-gamma globulin, and 0.35M NaCl; (b) PBS containing 0.5% bovine serum albumin (BSA), 0.05% P20, and 0.05% PROCLIN™ 300, pH 7; (c) PBS containing 0.5% BSA, 0.05% P20, 0.05% PROCLIN™ 300, 5 mM EDTA, and 0.35 M NaCl, pH 6.35; (d) PBS containing 0.5% BSA, 0.05% P20, 0.05% PROCLIN™ 300, 5 mM EDTA, 0.2% beta-gamma globulin, and 0.35 M NaCl; and (e) PBS containing 0.5% BSA, 0.05% P20, 0.05% PROCLIN™ 300, 5 mM EDTA, 0.25% CHAPS, and 0.35 M NaCl. Buffer (a) is the preferred buffer for the assay herein since it has the best differentiation between each standard as well as the biggest signal-to-noise ratio. PROCLIN™ 300 acts as a preservative, and TWEEN 20™ acts as a detergent to eliminate non-specific binding. The added EDTA and salt of buffer (a) act to decrease the background over the other buffers, including buffer (b).

The amount of capture reagents employed is sufficiently large to give a good signal in comparison with the standards, but not in molar excess compared to the maximum expected level of antibody of interest in the sample. For sufficient sensitivity, it is preferred that the amount of biological sample added be such that the immobilized capture reagents are in molar excess of the maximum molar concentration of free antibody of interest anticipated in the biological sample after appropriate dilution of the sample. This anticipated level depends mainly on any known correlation between the concentration levels of the free antibody of interest in the particular biological sample being analyzed with the clinical condition of the patient. Thus, for example, an adult patient may have a maximum expected concentration of free antibody of interest in his/her serum that is quite high, whereas a child will be expected to have a lower level of free antibody of interest in his/her serum based on the doses given.

While the concentration of the capture reagents will generally be determined by the concentration range of interest of the antibody of interest, taking any necessary dilution of the biological sample into account, the final concentration of the capture reagents will normally be determined empirically to maximize the sensitivity of the assay over the range of interest. However, as a general guideline, the molar excess is suitably less than about ten-fold of the maximum expected molar concentration of antibody of interest in the biological sample after any appropriate dilution of the sample.

The conditions for incubation of sample and immobilized capture reagent are selected to maximize sensitivity of the assay and to minimize dissociation, and to ensure that any antibody of interest present in the sample binds to the immobilized capture reagent. Preferably, the incubation is accomplished at fairly constant temperatures, ranging from about 0° C. to about 40° C., preferably at or about room temperature. The time for incubation is generally no greater than about 10 hours. Preferably, the incubation time is from about 0.5 to 3 hours, and more preferably about 1.5-3 hours at or about room temperature to maximize binding of the antibody of interest to the capture reagents. The duration of incubation may be longer if a protease inhibitor is added to prevent proteases in the biological fluid from degrading the antibody of interest.

At this stage, the pH of the incubation mixture will ordinarily be in the range of about 4-9.5, preferably in the range of about 6-9, more preferably about 7 to 8. The pH of the incubation buffer is chosen to maintain a significant level of specific binding of the capture reagents to the antibody of interest being captured. Various buffers may be employed to achieve and maintain the desired pH during this step, including borate, phosphate, carbonate, TRIS-HCl or TRIS-phosphate, acetate, barbital, and the like. The particular buffer employed is not critical to the invention, but in individual assays one buffer may be preferred over another.

Optional Second Step

In a second step of the assay method herein, which is optional, but preferred, the biological sample is separated (preferably by washing) from the immobilized capture reagents to remove uncaptured antibody of interest. The solution used for washing is generally a buffer (“washing buffer”) with a pH determined using the considerations and buffers described above for the incubation step, with a preferable pH range of about 6-9. The washing may be done three or more times. The temperature of washing is generally from refrigerator to moderate temperatures, with a constant temperature maintained during the assay period, typically from about 0-40° C., more preferably about 4-30° C. For example, the wash buffer can be placed in ice at 4° C. in a reservoir before the washing, and a plate washer can be utilized for this step. A cross-linking agent or other suitable agent may also be added at this stage to allow the now-bound antibody of interest to be covalently attached to the capture reagents if there is any concern that the captured antibody of interest may dissociate to some extent in the subsequent steps.

Third Step

In the next step, the immobilized capture reagents with any bound antibody of interest present are contacted with detectable antibody, preferably at a temperature of about 20-40° C., more preferably about 36-38° C., with the exact temperature and time for contacting the two being dependent primarily on the detection means employed. For example, when 4-methylumbelliferyl-β-galactoside (MUG), streptavidin-HRP, or streptavidin-β-galactosidase is used as the means for detection, preferably the contacting is carried out overnight (e.g., about 15-17 hours or more) to amplify the signal to the maximum. While the detectable antibody may be a polyclonal or monoclonal antibody, preferably it is a monoclonal antibody, more preferably rodent, still more preferably murine, yet still more preferably MAb 8A3 or 8C5, and most preferably MAb 8A3, to reduce background noise. Also, the preferred detectable antibody is directly detectable, and preferably is biotinylated. The detection means for the biotinylated label is preferably avidin or streptavidin-HRP, and the readout of the detection means is preferably fluorimetric or colorimetric.

Preferably, a molar excess of an antibody with respect to the maximum concentration of free antibody of interest expected (as described above) is added to the plate after it is washed. This antibody (which is directly or indirectly detectable) is preferably a monoclonal antibody, although any antibody can be employed. The affinity of the antibody must be sufficiently high that small amounts of the free antibody of interest can be detected, but not so high that it causes the antibody of interest to be pulled from the capture reagents.

The same anti-idiotypic antibody can be used for coat and detection in the assay, or different antibodies can be used for coat and detection. They are preferably selected so that the background noise is minimized.

Fourth Step

In the last step of the assay method, the level of any free antibody of interest from the sample that is now bound to the capture reagents is measured using a detection means for the detectable antibody. If the biological sample is from a clinical patient, the measuring step preferably comprises comparing the reaction that occurs as a result of the above three steps with a standard curve to determine the level of antibody of interest compared to the known amount.

The antibody added to the immobilized capture reagents will be either directly labeled, or detected indirectly by addition, after washing off of excess first antibody, of a molar excess of a second, labeled antibody directed against IgG of the animal species of the first antibody. In the latter, indirect assay, labeled antisera against the first antibody are added to the sample so as to produce the labeled antibody in situ.

The label used for either the first or second antibody is any detectable functionality that does not interfere with the binding of free antibody of interest to the anti-idiotypic antibodies. Examples of suitable labels are those numerous labels known for use in immunoassay, including moieties that may be detected directly, such as fluorochrome, chemiluminscent, and radioactive labels, as well as moieties, such as enzymes, that must be reacted or derivatized to be detected. Examples of such labels include the radioisotopes ³²P, ¹⁴C, ¹²⁵I, ³H, and 131I, fluorophores such as rare-earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, HRP, alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin (detectable by, e.g., avidin, streptavidin, streptavidin-HRP, and streptavidin-β-galactosidase with MUG), spin labels, bacteriophage labels, stable free radicals, and the like. The preferred label is biotin and the preferred detection means is avidin or streptavidin-HRP.

Conventional methods are available to bind these labels covalently to proteins or polypeptides. For instance, coupling agents such as dialdehydes, carbodiimides, dimaleimides, bis-imidates, bis-diazotized benzidine, and the like may be used to tag the antibodies with the above-described fluorescent, chemiluminescent, and enzyme labels. See, for example, U.S. Pat. Nos. 3,940,475 (fluorimetry) and 3,645,090 (enzymes); Hunter et al., Nature, 144:945 (1962); David et al., Biochemistry, 13:1014-1021 (1974); Pain et al., J. Immunol. Methods, 40:219-230 (1981); and Nygren, J. Histochem. and Cytochem., 30:407-412 (1982). The most preferred label herein is biotin using streptavidin-HRP for detection means.

The conjugation of such label, including the enzymes, to the antibody is a standard manipulative procedure for one of ordinary skill in immunoassay techniques. See, for example, O′Sullivan et al. “Methods for the Preparation of Enzyme-antibody Conjugates for Use in Enzyme Immunoassay,” in Methods in Enzymology, ed. J. J. Langone and H. Van Vunakis, Vol. 73 (Academic Press, New York, N.Y., 1981), pp. 147-166.

Following the addition of last labeled antibody, the amount of bound antibody is determined by removing excess unbound labeled antibody through washing and then measuring the amount of the attached label using a detection method appropriate to the label, and correlating the measured amount with the amount of the antibody of interest in the biological sample. For example, in the case of enzymes, the amount of color developed and measured will be a direct measurement of the amount of the antibody of interest present. Specifically, if HRP is the label, the color is detected using the substrate OPD at 490-nm absorbance.

In one example, after an enzyme-labeled second antibody directed against the first unlabeled antibody is washed from the immobilized phase, color or chemiluminiscence is developed and measured by incubating the immobilized capture reagent with a substrate of the enzyme. Then the concentration of the antibody of interest is calculated by comparing with the color or chemiluminescence generated by the standard antibody of interest run in parallel.

Antibody Production

A description follows as to exemplary techniques for the production of the anti-idiotypic antibodies used in accordance with the present invention.

(i) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor, using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOC1₂, or R¹N=C=NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(ii) Monoclonal Antibodies

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies.

For example, the monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2, P3X63Ag.U.1, or X63-Ag8-653 cells available from the American Type Culture Collection, Manassas, Va. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antibody of interest. Preferably, the binding specificity of monoclonal antibodies produced by hybridoma cells is determined by immunoprecipitation or by an in vitro binding assay, such as radioimmunoassay (RIA) or ELISA. Such clones are also screened for those that produce the least background noise in the assay when used as capture reagents and/or detectable antibodies

The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-SEPHAROSE™ agarose chromatography, hydroxylapatite chromatography, gel electrophoresis, dialysis, or affinity chromatography.

One specific preparation technique using hybridoma technology comprises immunizing mice such as CAF1 mice or Balb/c, for example, by injection in the footpads or spleen, with the antibody of interest in an adjuvant such as monophosphoryl lipid A/trehalose dicorynomycolate or as a conjugate of the antibody of interest with keyhole limpet haemocyanin (KLH) or with Limulus hemocyanin. Injections are done as many times as needed. The mice are sacrificed and popliteal lymph nodes or splenocytes obtained from the immunized mice, especially those with high titers, are fused with a murine myeloma cell line such as SP2/0 or P3X63Ag.U.1 (American Type Culture Collection (ATCC, Manassas, Va.)).

The resulting hybridomas are screened for antibodies with binding affinity for the antibody of interest but not other antibodies binding to a different antigen. This screening may take place by conventional ELISA for secretion of antibody that binds to immobilized antibody of interest or for production of IgG with an inhibition capacity of more than about 95% (inhibition of binding of the antibody of interest to the protein antigen). This screen defines a population of antibodies with nominal or higher reactivity as well as selectivity for the antibody of interest. Further selection may be performed to identify those antibodies with properties especially preferred for ELISAs. The criteria used for selecting a preferred anti-idiotypic antibody include that it should bind to the antibody of interest with relatively high affinity (Kd<about 10⁻⁸M), and its binding to the antibody of interest should be mutually exclusive with binding to the analyte transmembrane protein. It should also provide the cleanest assay with the least background noise.

The positive clones may be re-screened using surface plasmon resonance using a BIACORE™ instrument to measure the affinity of the anti-idiotypic antibody for the antibody of interest (as reflected in its off-rate) and the mutual exclusivity of binding. Rabbit anti-mouse IgG(Fc) may be immobilized onto the biosensor surface and used to capture anti-idiotypic antibodies from hybridoma culture supernates. The antibody of interest at 0.2 nM alone and in the presence of 0.9 nM C-reactive protein (CRP) may be injected over the surface of the immobilized anti-idiotypic antibody and the relative mass accumulation compared. The hybridoma cells that are selected are cloned as by limiting dilution to obtain the desired clones. The anti-idiotypic antibody can then be purified and isolated from these clones. See U.S. Pub. No. US 20020142356 for an example of preparing an anti-idiotypic antibody, as well as Durrant et al., Int J. Cancer, 1:92(3):414-20 (2001) and Bhattacharya-Chatterjee, Curr. Opin. Mol. Ther., 3(1):63-9 (2001).

The monoclonal antibodies may also be produced recombinantly. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs., 130:151-188 (1992).

In a further embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high-affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin-coding sequence all or part of the coding sequence for a non-immunoglobulin polypeptide.

Many of the procedures useful for practicing the present invention, whether or not described herein in detail, are well known to those skilled in the arts of molecular biology, biochemistry, immunology, and medicine. Once the antibody of interest is identified, generating the anti-idiotypic antibody would be within the skill of the ordinarily skilled practitioner in this field.

Kits

As a matter of convenience, the assay method of this invention can be provided in the form of a kit. Such a kit is a packaged combination including the basic elements of:

(a) capture reagents comprised of anti-idiotypic antibodies against the antibody of interest, wherein the antibodies bind specifically to two different binding sites on the antibody of interest;

(b) detectable (labeled or unlabeled) anti-idiotypic antibodies that bind specifically to two different binding sites on the antibody of interest; and

(c) instructions on how to perform the assay method using these reagents. These basic elements are defined hereinabove.

Preferably, the kit further comprises a solid support for the capture reagents, which may be provided as a separate element or on which the capture reagents are already immobilized. Hence, the capture antibodies in the kit may be immobilized on a solid support, or they may be immobilized on such support that is included with the kit or provided separately from the kit. Preferably, the capture reagents are coated on a microtiter plate. The detectable antibodies may be labeled antibodies detected directly or unlabeled antibodies that are detected by labeled antibodies directed against the unlabeled antibodies raised in a different species. Where the label is an enzyme, the kit will ordinarily include substrates and cofactors required by the enzyme, where the label is a fluorophore, a dye precursor that provides the detectable chromophore, and where the label is biotin, an avidin such as avidin, streptavidin, or streptavidin conjugated to HRP or β-galactosidase with MUG.

In a preferred specific embodiment, the capture reagents are monoclonal antibodies, preferably rodent, more preferably murine or rat, still more preferably murine, and most preferably MAb 8A3 or MAb 8C5. Also in preferred embodiments, the detectable antibody is a biotinylated monoclonal antibody, the monoclonal antibody is rodent, more preferably murine or rat, still more preferably murine, yet still more preferably MAb 8A3 or MAb 8C5, and most preferably MAb 8A3. Preferably, the capture reagents are immobilized in this kit.

The kit also typically contains the antibody of interest as a standard (e.g., purified antibody of interest), as well as other additives such as stabilizers, washing and incubation buffers, and the like.

Examples of standards for the antibody of interest are monoclonal antibodies, more preferably humanized antibodies, and still more preferably a humanized 2H7 antibody such as available from Genentech, Inc., South San Francisco, Calif.

The components of the kit will be provided in predetermined ratios, with the relative amounts of the various reagents suitably varied to provide for concentrations in solution of the reagents that substantially maximize the sensitivity of the assay. Particularly, the reagents may be provided as dry powders, usually lyophilized, including excipients, which on dissolution will provide for a reagent solution having the appropriate concentration for combining with the sample to be tested.

### III. EXPERIMENTAL EXAMPLES

The above and other features of the invention will now be described more particularly with reference to the accompanying figures and pointed out in the claims. The particular embodiments described below are provided by way of illustration and are not meant to be construed as a limitation on the scope of the invention. It will be apparent to one of ordinary skill in the art that many modifications can be made to the present invention without departing from the spirit or essential characteristics of the invention. The following examples are intended to illustrate embodiments now known for practicing the invention, but the invention is not to be considered limited to these examples. The disclosures of all citations herein are expressly incorporated by reference.

### Example 1

Materials and Methods

Anti-CD20 Antibody

Full-length chimeric antibody and humanized anti-CD20 antibody variants were generated from a mouse anti-human CD20 antibody using a human IgG₁, framework at Genentech, Inc. They were expressed in 293 cells and purified using a protein A column as described previously (Presta et al., Cancer Res., supra). See FIGS. 6A and 6B for the amino acid sequences of the respective light-and heavy-chain variable domains (V_{L} and V_{H}) of the parent murine antibody, humanized variant h2H7.v16 (SEQ ID NO: 12), and the human kappa light chain of subgroup I or the human consensus sequence of heavy-chain subgroup III.

CD20-Expressing CHO Clones

Human CD20 cDNA (Genentech, Inc.) was subcloned into a modified dihydrofolate reductase (DHFR) intron vector at the SpeI site as described in Meng et al., Gene, 242: 201-207 (2000). CHO K1 DUX B 11 (DHFR-) cells (Columbia University) were grown in 50:50 F12/DMEM medium supplemented with 2 mM L-glutamine, 10 μg/ml glycine, 15 μg/ml hypoxanthine, 5 μg/ml thymidine, 100 units/ml penicillin, 100 μg/ml streptomycin, and 5% fetal bovine serum (FBS) (Gibco BRL Life Technologies, Gaithersburg, Md) in a humidified 5% CO₂ incubator at 37° C. CHO cells in 100-mm diameter plates were transfected with a 4 μg/ml linearized plasmid vector using POLYFECT™ transfection system (Qiagen Inc., Santa Clarita, Calif.) following the manufacturer's instructions. Transfected CHO cells were grown in 50:50 F12/DMEM medium supplemented with 2 mM L-glutamine, 100 units/ml penicillin, 100 μg/ml streptomycin and 5% dialyzed FBS. Clones with different CD20 expression levels were obtained by repeated fluorescence-activated cell sorter (FACS) sorting as described by Meng et al., supra, using 5 μg/ml RITUXAN® followed by fluorescein isothiocyanate (FITC)-conjugated goat anti-human IgG Fc (Jackson ImmunoResearch Laboratories, West Grove, Pa.) for staining. Clone C12M was obtained by growing clone 2H3 cells in 25-nM methotrexate.

WIL2 Binding Assay

Human B-lymphoblastoid WIL2-S cells (American Type Culture Collection, Manassas, Va.) were grown in RPMI 1640 supplemented with 2 mM L-glutamine, 20 mM HEPES, pH 7.2, and 10% heat-inactivated FBS in a humidified 5% CO₂ incubator at 37° C. They were washed with PBS containing 1% FBS (assay buffer) and seeded at 250,000-300,000 cell/well in 96-well round-bottom plates (Nunc, Roskilde, Denmark). Standards (15.6-1000 ng/ml of chimeric anti-CD20 IgG in twofold serial dilutions) and samples (2.7-2000 ng/ml of humanized anti-CD20 IgG in threefold serial dilutions) in 100-μl assay buffer were added to the plates. The plates were incubated on ice for 45 min. To remove the unbound antibody, 100 μl of assay buffer was added to the wells. Plates were centrifuged and supernatants were removed. Cells were washed two more times with 200 μl of assay buffer. Bound antibody was detected by adding HRP-conjugated goat anti-human IgG Fc antibody (Jackson ImmunoResearch, West Grove, Pa.) to the plates. After a 45-min incubation on ice, cells were washed and the substrate 3,3′,5,5′-tetramethyl benzidine (Kirkegaard & Perry Laboratories, Gaithersburg, Md.) was added. The reaction was stopped by adding 1 M phosphoric acid. Absorbance was read at 450 nm on a TITERTEK™ stacker reader (ICN, Costa Mesa, Calif.). Titration curves were fit with a four-parameter regression curve-fitting program (KALEIDAGRAPH™ software, Synergy Software, Reading, Pa.). The absorbance at the midpoint of the titration curve (mid-OD) of standard was calculated. The corresponding concentrations of standard and samples at this mid-OD were determined (KALEIDAGRAPH™ software). The relative activity was calculated by dividing the mid-OD concentration of standard by that of sample. Coefficient of variation (CV) by ANOVA analysis was calculated using the STATVIEW™ program (SAS Institute, Cary, N.C.). Values shown were mean ±standard deviation. Error bars in figures were standard deviations.

CHO Binding Assay

The assay was performed similarly as the WIL2 binding assay unless mentioned otherwise. For the suspension format, CHO cells were detached using a non-enzymatic cell-dissociation solution (Sigma, St. Louis, Mo.). For the adherent format, 2H3 CHO cells were grown in flat-bottom 96-well cell-culture plates (Falcon, Becton Dickinson Labware, Franklin, N.J.) and were 80-90% confluent on the day of the assay. Growth medium was used for the assay in order to keep the cells attached to the plates. Cells were washed between incubation steps by adding the growth medium to the plates and flicking the plates to remove the wash buffer.

Scatchard Analysis

RITUXAN®(Genentech Inc., South San Francisco, Calif. and IDEC Pharmaceuticals, San Diego, Calif.; Reff et al., Blood, 83: 435-445 (1994)) was iodinated using the lactoperoxidase method (13.7 mCi/mg). For the adherent format, CHO cells were seeded onto 24-well plates at 50,000 cells/well. After a two-day growth, 0.2 nM labeled RITUXAN™ and 2.5-fold serially diluted non-labeled RITUXAN®(10-1000 nM) in 0.4-ml F12/DMEM 50:50, 2% FBS (binding buffer) was added to the cells. After a two-hour incubation on ice, cells were washed with the binding buffer and detached using trypsin-EDTA (CLONETICS®, Cambrex Bio Science Walkersville, Inc., Walkersville, Md.) and counted in a gamma counter (Packard Instrument Company, Perkin-Elmer, Downers Grove, Ill.). The number of cells per well used for data analysis was determined by counting the cells in control wells not receiving RITUXAN®. For the suspension format, cells were detached using non-enzymatic cell-dissociation solution (Sigma). Labeled and non-labeled RITUXAN® antibodies were incubated with 300,000 cells in 1.5-ml conical test tubes as described above. Cells were centrifuged and washed with 0.8 ml FBS. They were suspended in 0.5 ml PBS and counted as described above. Binding constants and number of receptors were calculated using the NEW LIGAND™ program (Genentech, Inc.) written according to the LIGAND™ program (Munson and Rodbard, Anal. Biochem., 107: 220-239 (1980)).

Generation of Specific Anti-Idiotypic Antibodies

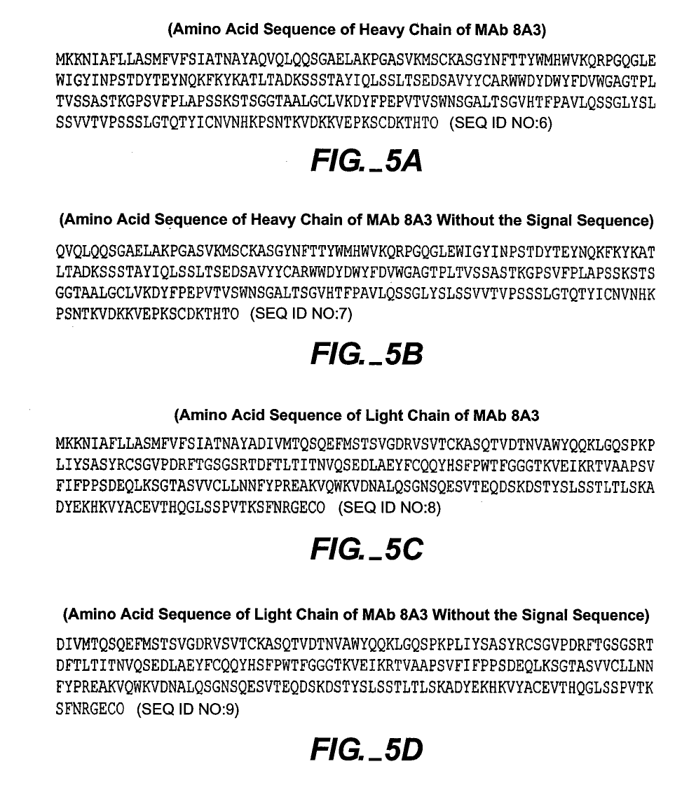

Monoclonal antibodies to a humanized anti-CD20 antibody were generated by injecting 0.5 μg of a humanized anti-CD20 IgG (2H7.v16 shown in FIG. 6) in monophosphoryl lipid A/trehalose dicorynomycolate adjuvant (Corixa, Hamilton, Mont.) in the footpads of Balb/c mice (Charles River Laboratories, Wilmington, Del.) eleven times. Popliteal lymph nodes from mice with high titers were fused with P3X63Ag.U. 1 myeloma cells (American Type Culture Collection (ATCC, Manassas, Va.)). Hybridoma cells producing antibodies with binding affinity for humanized anti-CD20 IgG, but not HERCEPTIN®, were cloned by limiting dilution to obtain clones 8C5 and 8A3. These hybridomas, called 8C5.1 and 8A3.10, are deposited as ATCC Nos. PTA-5915 and PTA-5914, producing these antibodies, respectively. The sequence of antibody 8A3 is provided in FIG. 5.

ELISA for Quantification of Anti-CD20 Antibodies.

MAXISORP™96-well microwell plates (Nunc, Roskilde, Denmark) were coated overnight at 4° C. with 0.25 μg/ml anti-idiotypic antibody 8C5 in 50 mM carbonate buffer, pH 9.6. Plates were blocked with 0.5% bovine serum albumin, 10 ppm PROCLIN 300™(Supelco, Bellefonte, Pa.) in PBS. Humanized anti-CD20 IgG or the parent mouse anti-CD20 IgG standards (2.0-250 ng/ml in 2-fold serial dilution) in PBS containing 0.5% bovine serum albumin, 0.05% POLYSORBATE 20™ non-ionic surfactant, 5 mM EDTA, 0.25% CHAPS, 0.2% bovine gamma-globulins (Sigma, St. Louis, Mo.) and 0.35N NaCl (sample buffer) were added to the plates. After a 2-hour incubation at room temperature, antibody bound to the plates was detected by adding biotinylated 8A3 followed by streptavidin-HRP (Amdex, Copenhagen, Denmark). Plates were developed and the titration curve of standard was fitted as described above. Data points that fell in the range of the standard curve were used for calculating the anti-CD20 antibody concentrations in samples. Serum effects were studied. using pooled mouse or human serum (Golden West Biologicals Inc., Temecula, Calif.).

Results

Cell-Binding Assays for Measuring Relative Binding Affinity of Humanized Anti-CD20 Antibodies

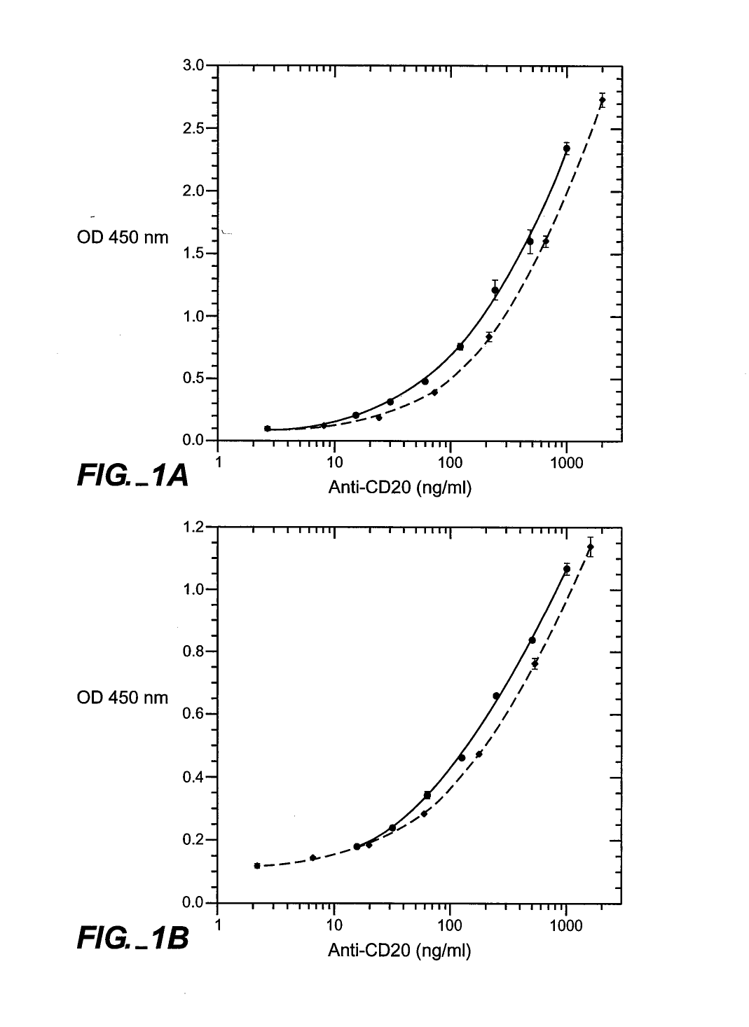

A WIL2 binding assay was developed to measure relative binding affinity of humanized anti-CD20 antibody variants, since CD20 is a multi-transmembrane protein and a native soluble CD20 extracellular was not available. In this assay, WIL2 cells were incubated with serially diluted anti-CD20 antibodies and bound anti-CD20 antibody was detected using anti-human IgG Fc-HRP. Cells were washed between incubation steps by adding wash buffer, centrifuging the cells, and removing the wash buffer. This assay was quantitative and reproducible. Representative titration curves of a humanized anti-CD20 IgG and the chimeric anti-CD20 antibody derived from the same parent mouse antibody are shown in FIG. 1A This humanized anti-CD20 IgG was assayed in 12 independent assays in duplicate and the relative binding activity to the chimeric anti-CD20 IgG was 0.63±0.08. The inter- and intra-assay CVs were 11.2% and 8.77%, respectively.

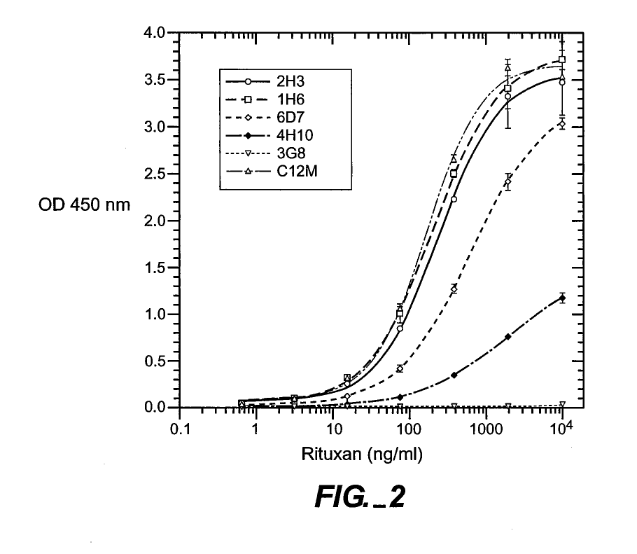

Also evaluated was a cell-binding assay using adherent transfected CHO cells in order to simplify the wash steps and increase the assay throughput. Representative titration curves of the chimeric anti-CD20 IgG and humanized anti-CD20 IgG binding to a high-expression CHO clone 2H3 are shown in FIG. 1B. Signals were lower than that obtained using the WIL2 cells (FIG. 1A), likely due, without being limited to any one theory, to two-fold fewer cells used in the adherent format. Several humanized antibody variants were assayed in both the WIL2 and CHO 2H3 binding assays and similar results were obtained. Since it took time to amplify cells to obtain high-expression clones, the minimum number of CD20 molecules per cell required for generating a good titration curve was tested. CHO clones expressing different levels of CD20 were obtained by FACS sorting. Selected clones were evaluated for binding to RITUXAN® (FIG. 2) and analyzed by Scatchard analysis (Table 1).

The number of CD20 molecules was estimated to be 1.2 million per cell for clone 2H3 using the adherent cell format. The numbers of CD20 molecules were estimated to be 1.0 and 0.16 million per cell for WIL2 and clone 2H3, respectively, using the suspension cell format. The binding affinities for 2H3 CHO and WIL2 cells were estimated to be 8.6 and 3.9 nM, respectively (Table 1). These affinities were close to the estimated 5.2 nM binding affinity of RITUXAN® for human SB cells (Reff et al., supra). CHO clone 4H10 expressing as few as 33,000 CD20 molecules per cell gave a good titration curve in the binding assay (Table 1 and FIG. 2). This level of expression is within two-fold of the expression of 60,000 CD20 molecules per cell found on Daudi cells (Bubien et al., J. Cell. Biol., 121: 1121-1132 (1993)) and may be sufficient for evaluating anti-CD20 antibodies in general.

TABLE 1
Scatchard analysis of CD20 expressing cells (n = 3)

| | | | | | Standard |
|---|---|---|---|---|---|
| | | CD20 copy^{a} | Standard error | Kd | error |
| Format | Clone | (million/cell) | (million/cell) | (nM) | (nM) |
| Adherent | 2H3 | 1.22 | 0.06 | 12.0 | 1.0 |
| | 1H6 | 1.28 | 0.05 | 11.5 | 0.8 |
| | 6D7 | 0.189 | 0.007 | 5.97 | 0.40 |
| | C12M | 1.31 | 0.06 | 13.7 | 1.0 |
| | 4H10 | 0.0332 | 0.0050 | 5.50 | 1.10 |
| Suspension | 2H3 | 1.00 | 0.08 | 8.57 | 0.97 |
| | WIL2 | 0.163 | 0.012 | 3.91 | 0.40 |

^{a}Calculated assuming one antibody binds one CD20 molecule.

Anti-Idiotypic Antibody Binding Assay for Measuring Serum Concentrations of Humanized Anti-CD20 Antibody

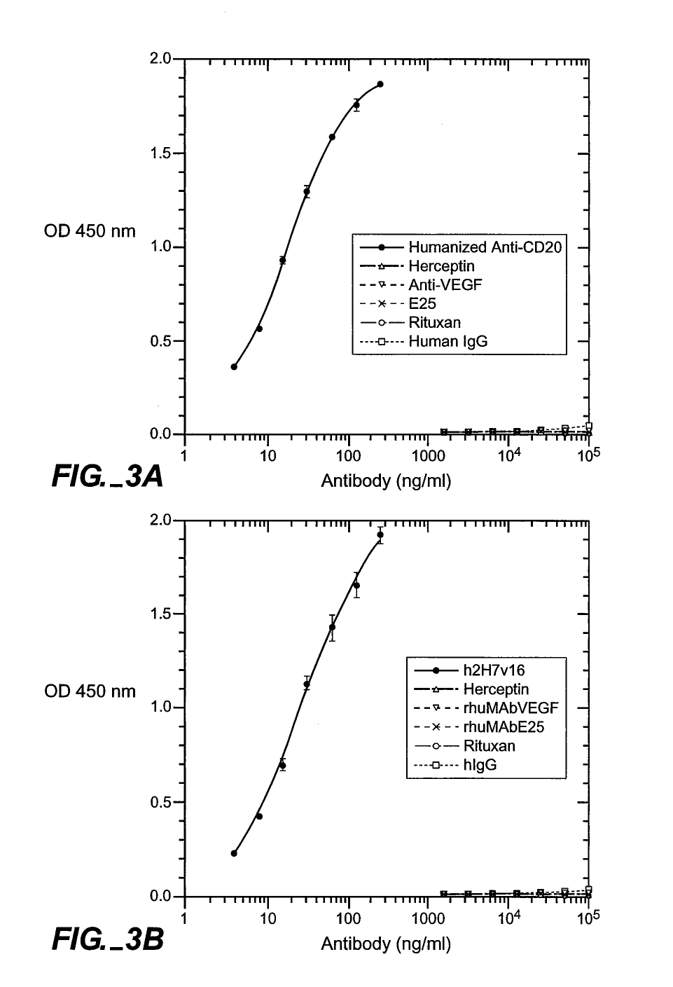

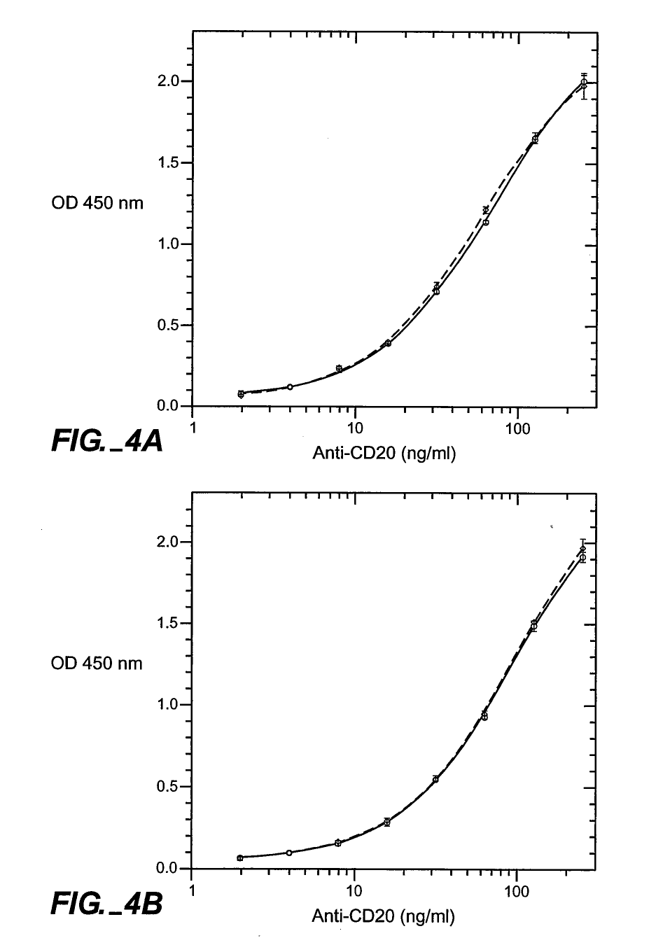

For measuring serum concentrations of humanized anti-CD20 antibody for clinical studies, an alternative approach involving a high-throughput assay was developed using specific anti-idiotypic antibodies to the humanized anti-CD20 antibody 2H7.v16, since a native CD20 molecule was not required. Antibodies 8C5 and 8A3 blocked the binding of the humanized 2H7 (2H7.v16) and chimeric 2H7 anti-CD20 antibody, but not RITUXAN®, to WIL2 cells. When coated on plates, they bound to humanized anti-CD20 IgG (2H7.v16 and 2H7.v31-see FIGS. 6 and 8 for sequences), but not HERCEPTIN®, E25, and anti-VEGF, which were humanized using the same human IgG, framework. They also showed no binding to RITUXAN® and little binding (<50,000 fold) to normal human IgG (FIG. 3). An ELISA using 8C5 for coat and biotinylated 8A3 for detection tolerated 20% human serum well. The recovery of 3.9-250 ng/ml humanized anti-CD20 IgG in 20% human serum was 93-117% (FIG. 4A). Therefore, this assay had a sensitivity of 20 ng/ml for humanized anti-CD20 IgG in human serum and can be used to support clinical studies.

Anti-idiotypic antibodies 8C5 and 8A3 also recognized the parent mouse anti-CD20 antibody used for humanization. The parent mouse anti-CD20 IgG gave a good titration curve in the ELISA using 8C5 for coat and biotinylated 8A3 for detection. The recovery of 2.0-250 ng/ml mouse anti-CD20 IgG in 10% mouse serum was 97-109% (FIG. 4B). The reproducibility of the assay was evaluated using a mouse anti-CD20 IgG that had the same variable domain as the parent mouse anti-CD20 antibody. Frozen aliquots of high, middle and low controls in sample buffer were assayed with the standards and their concentrations were 96.1±6.5, 17.4±1.2 and 2.26±0.69 ng/ml, respectively. The percent CV for the high, middle, and low controls in buffer were 4.56, 7.06, and 29.3 for the inter-assay, respectively, and 7.05, 2.58, and 13.8 for the intra-assay, respectively (n=12). The low control had a concentration close to the 2.0 ng/ml concentration of the lowest standard and had higher assay variations.

Discussion

For quantification of serum concentrations of humanized anti-CD20 antibody for clinical studies, the effect of human serum on WIL2 and CHO binding assays was assayed. In the WIL2 binding assay, the presence of 10% human serum gave a background equivalent to 100 ng/ml of humanized anti-CD20 IgG and reduced the signal. In the CHO binding assay, it did not give a significant background but greatly reduced the signal. Signal reduction was also seen in an ELISA using a membrane preparation of WIL2 cells for coat. Without being limited to any one theory, this signal reduction may be due to circulating human CD20 in serum (Manshouri et al., Blood, 101:2507-2513 (2003)). The presence of 10% mouse serum did not affect the WIL2 binding assay significantly. The recovery for 16-1000 ng/ml humanized anti-CD20 IgG in 10% mouse serum was 75-102%. Since it was not necessary to use a native CD20 molecule, an antibody to the intracellular domain of CD20 (clone 1H1 (FB 1), BD PharMingen, San Diego, Calif.) was used to capture CD20 in the lysed WIL2 cells, but this did not result in sufficient assay sensitivity.

As an alternative, improved method, an ELISA using specific anti-idiotypic antibodies, namely, 8C5 for coat and biotinylated 8A3 for detection, was developed for quantification of humanized anti-CD20 antibody in human serum (FIG. 4A). Since antibody 8C5 had a slight affinity for normal human IgG (FIG. 3A) and human IgG was present at a high concentration in human serum, 20% human serum gave a background equivalent to 4 ng/ml humanized anti-CD20 IgG when anti-human IgG Fc-HRP was used for detection. Therefore, the use of biotinylated 8A3 for detection was important for reducing the serum background. The detection antibody 8A3 in solution competed with 8C5 coated on the plate for binding to humanized anti-CD20 IgG (v.16). However, since IgG has two binding sites and can bind to one 8C5 and one 8A3 at the same time, humanized anti-CD20 IgG gave a good titration curve in this ELISA. Coating with 0.25 μg/ml 8C5 gave higher signals compared to coating with 1 μg/ml. Without being limited to any one theory, it is believed that at a lower coating density, humanized anti-CD20 IgG was more likely to bind to the 8C5-coated plate with only one binding site, allowing the other binding site to bind to the detection antibody 8A3. The ELISA using 8C5 for coat and biotinylated 8A3 for detection could also be used for measuring the parent mouse anti-CD20 antibody in mouse serum for xenograft or other mouse studies (FIG. 4B). The WIL2 binding assay using anti-mouse Fc-HRP for detection could not be used for this purpose since 10% mouse serum gave a high background.

Serum concentrations of a mouse anti-CD20 antibody that had the same variable domain as the parent mouse anti-CD20 antibody were measured by this ELISA. The results agreed with that obtained by a less sensitive ELISA using 8A3 Fab for coat and anti-mouse IgG Fc-HRP for detection, which did not compete with the coat antibody. This mouse anti-CD20 antibody also gave a good titration curve in an ELISA using 8A3 for coat and biotinylated 8A3 for detection. Therefore, it is possible to develop an ELISA for anti-CD20 antibody using only one specific anti-idiotypic antibody, with similar results being obtained for both.

### Example 2

An ELISA as set forth in Example 1 can be employed to detect antibodies to a chemokine receptor. This would be useful, for example, to detect humanized antibodies to a chemokine receptor in a clinical sample, where the humanized antibodies are administered to clinical patients to treat a chemokine-mediated disorder. Thus, anti-idiotypic monoclonal antibodies are generated to murine MAb LS 132.1D9 (1D9) or to a humanized antibody that can compete with 1D9 for binding to human CCR2 as described in U.S. Pat. No. 6,696,550, by injecting 0.5 μg of 1D9 or the humanized antibody formulated in monophosphoryl lipid A/trehalose dicorynomycolate adjuvant (Corixa, Hamilton, Mont.) into the footpads of Balb/c mice (Charles River Laboratories, Wilmington, Del.) eleven times. Popliteal lymph nodes from mice with high titers are fused with P3X63Ag.U.1 myeloma cells (American Type Culture Collection (ATCC, Manassas, Va.)). Hybridoma cells producing antibodies with binding affinity for 1D1 or the humanized antibody used as immunogen, but not for other mouse antibodies of the same subclass as 1D1 or other humanized antibody, that was humanized using the same framework, directed to a different epitope or antigen, are cloned by limiting dilution to obtain suitable clones. The antibodies from such clones, which are anti-idiotypic to 1D1 or the humanized antibody used as immunogen, are isolated from the clones and used as coat and detection means in a biological sample containing or suspected of containing 1D1 or the humanized antibody used as immunogen, using the basic ELISA method disclosed in Example 1.

Alternatively, MAb 3C3, which selectively reacts with GPR-9-6 transfectants (see U.S. Pat. No. 6,689,570) is used to immunize the balb/c mice using the technique as noted above to obtain anti-idiotypic antibodies to MAb 3C3, which are then used in the assay as coat and detection agents.

In summary, an anti-idiotypic-antibody-based assay has been developed for measuring concentrations, in biological samples such as serum, of an antibody of interest, for example, a humanized antibody and its parent mouse antibody or the chimeric murine/human antibody derived from the parent antibody. This anti-idiotypic-antibody-based approach may be applied in general for detecting and measuring in biological samples the antibodies or the concentrations of antibodies directed to cell-surface transmembrane proteins with a small intervening extracellular domain such as CD20 and chemokine receptors.

### Example 3

### Preparation of Humanized Antibodies

The humanized 2H7 antibody may comprise one, two, three, four, five, or six of the following CDR sequences:
CDR L1 sequence RASSSVSYXH wherein X is M or L (SEQ ID NO:29), for example SEQ ID NO: 14 (FIG. 6A),
CDR L2 sequence of SEQ ID NO: 15 (FIG. 6A),
CDR L3 sequence QQWXFNPPT wherein X is S or A (SEQ ID NO:30), for example SEQ ID NO: 16 (FIG. 6A),
CDR H1 sequence of SEQ ID NO:20 (FIG. 6B),
CDR H2 sequence of AIYPGNGXTSYNQKFKG where X is D or A (SEQ ID NO:31), for example SEQ ID NO:21 (FIG. 6B), and
CDR H3 sequence of VVYYSXXYWYFDV where X at position 6 is N, A, or Y, and X at position 7 is S or R (SEQ ID NO:32), for example SEQ ID NO:22 (FIG. 6B).

The CDR sequences above are generally present within human variable light and variable heavy framework sequences, such as substantially the human consensus FR residues of human light-chain kappa subgroup I (V_{L}KI), and substantially the human consensus FR residues of human heavy-chain subgroup III (V_{H}III).

The variable heavy region may be joined to a human IgG chain constant region, wherein the region may be, for example, IgG1 or IgG3. See also WO2004/056312 (Lowman et al.).

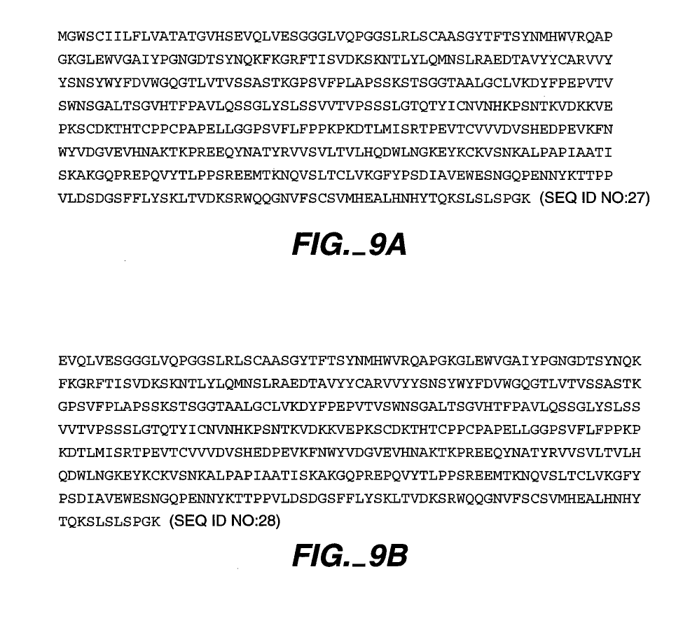

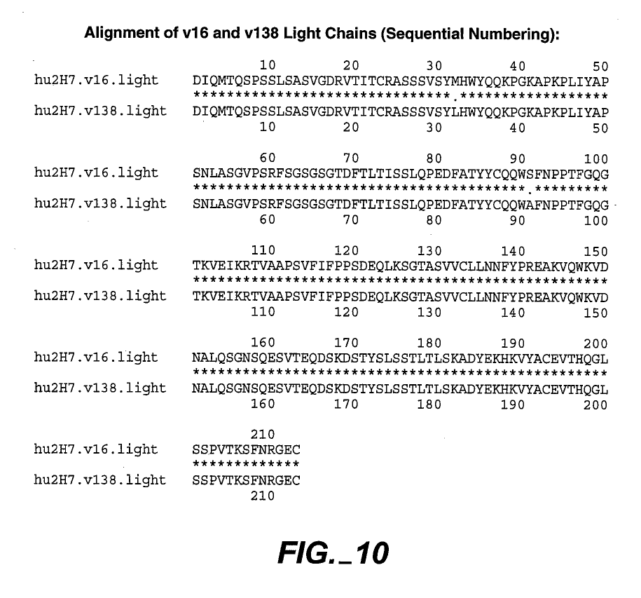

In a preferred embodiment, such antibody comprises the variable heavy-domain sequence of SEQ ID NO: 18 (v16, as shown in FIG. 6B), optionally also comprising the variable light-domain sequence of SEQ ID NO: 12 (v16, as shown in FIG. 6A), which optionally comprises the amino acid substitutions of D56A and N100A in the heavy chain and S92A in the light chain (v96). Preferably, the antibody is an intact antibody comprising the light- and heavy-chain amino acid sequences of SEQ ID NOS:3 and 4 or 5, respectively. A preferred humanized 2H7 antibody is ocrelizumab. The antibody herein may further comprise at least one amino acid substitution in the Fc region that improves ADCC activity, such as one wherein the amino acid substitutions are at positions 298, 333, and 334, preferably S298A, E333A, and K334A, using EU numbering of heavy chain residues. Another preferred embodiment is where the antibody is 2H7.v138 comprising the light-chain and heavy-chain amino acid sequences of SEQ ID Nos. 33 and 34, respectively, as shown in FIGS. 10 and 11, which are alignments of such sequences with the corresponding light-chain and heavy-chain amino acid sequences of 2H7.v16. Alternatively, such preferred intact humanized 2H7 antibody is 2H7.v477, which has the light-chain and heavy-chain sequences of 2H7.v138 except for the amino acid substitution at heavy-chain position 434, for example, N434W, which increases FcRn binding and serum half-life of the antibody. Any of these antibodies may further comprise at least one amino acid substitution in the Fc region that increases CDC activity, for example, comprising at least the substitution at position 326, preferably K326A. See U.S. Pat. No. 6,528,624B1 (Idusogie et al.).

Some preferred humanized 2H7 variants are those comprising the variable light domain of SEQ ID NO: 12 and the variable heavy domain of SEQ ID NO: 18, including those with or without substitutions in an Fc region (if present), and those comprising a variable heavy domain with alteration N100A; or D56A and N100A; or D56A, N100Y, and S100aR; in SEQ ID NO:18 and a variable light domain with alteration M32L; or S92A; or M32L and S92A; in SEQ ID NO: 12.

In a summary of some various preferred embodiments of the invention, the variable region of variants based on 2H7.v16 will have the amino acid sequences of v16 except at the positions of amino 10 acid substitutions that are indicated in Table 2 below. Unless otherwise indicated, the 2H7 variants will have the same light chain as that of v16.

TABLE 2
2H7 Variants

| | Heavy chain | Light chain | |
|---|---|---|---|
| 2H7 version | (V_{H}) changes | (V_{L}) changes | Fc changes |
| 16 for | | | — |
| reference | | | |
| 31 | — | — | S298A, E333A, K334A |
| 73 | N100A | M32L | |
| 75 | N100A | M32L | S298A, E333A, K334A |
| 96 | D56A, N100A | S92A | |
| 114 | D56A, N100A | M32L, S92A | S298A, E333A, K334A |
| 115 | D56A, N100A | M32L, S92A | S298A, E333A, K334A, |
| | | | E356D, M358L |
| 116 | D56A, N100A | M32L, S92A | S298A, K334A, K322A |
| 138 | D56A, N100A | M32L, S92A | S298A, E333A, K334A, |
| | | | K326A |
| 477 | D56A, N100A | M32L, S92A | S298A, E333A, K334A, |
| | | | K326A, N434W |
| 375 | — | — | K334L |
| 588 | — | | S298A, E333A, K334A, |
| | | | K326A |
| 511 | D56A, N100Y, | | S298A, E333A, K334A, |
| | S100aR | | K326A |

A particularly preferred humanized 2H7 is an intact antibody or antibody fragment comprising the variable light-domain sequence: DIQMTQSPSSLSASVGDRVTITCRASSSVSYMHWYQQKPGKAPKPLIYAPSNLASGVPSRFSG SGSGTDFTLTISSLQPEDFATYYCQQWSFNPPTFGQGTKVEIKR (SEQ ID NO:12);

and the variable heavy-domain sequence: EVQLVESGGGLVQPGGSLRLSCAASGYTFTSYNMHWVRQAPGKGLEWVGAIYPGNGDTSY NQKFKGRFFISVDKSKNTLYLQMNSLRAEDTAVYYCARVVYYSNSYWYFDVWGQGTLVTV SS (SEQ ID NO:18).

Where the humanized 2H7 antibody is an intact antibody, it may comprise the light-chain amino acid sequence:

| | |
|---|---|
| DIQMTQSPSSLSASVGDRVTITCRASSSVSYMHWYQQKPGKAPKPLIYAPSNLASGVPSRFSG | (SEQ ID NO: 3) |
| SGSGTDFTLTISSLQPEDFATYYCQQWSFNPPTFGQGTKVEIKRTVAAPSVFIFPPSDEQLKSG | |
| TASVVCLLNNFYPREAKVQWKVDNALQSGNSQESVTEQDSKDSTYSLSSTLTLSKADYEKH | |
| KVYACEVTHQGLSSPVTKSFNRGEC; | |

and the heavy-chain amino acid sequence:

| | |
|---|---|
| EVQLVESGGGLVQPGGSLRLSCAASGYTFTSYNMHWVRQAPGKGLEWVGAIYPGNGDTSY | (SEQ ID NO: 4) |
| NQKEKGRFTISVDKSKNTLYLQMNSLRAEDTAVYYCARVVYYSNSYWYFDVWGQGTLVTV | |
| SSASTKGPSVFPLAPSSKSTSGGTAALGCLVKDYFPEPVTVSWNSGALTSGVHTFPAVLQSSG | |
| LYSLSSVVTVPSSSLGTQTYICNVNHKPSNTKVDKKVEPKSCDKTHTCPPCPAPELLGGPSVF | |
| LFPPKPKDTLMISRTPEVTCVVVDVSHEDPEVKENWYVDGVEVHNAKTKPREEQYNSTYRV | |
| VSVLTVLHQDWLNGKEYKCKVSNKALPAPIEKTISKAKGQPREPQVYTLPPSREEMTKNQVS | |
| LTCLVKGFYPSDIAVEWESNGQPENNYKTTPPVLDSDGSFFLYSKLTVDKSRWQQGNVFSCS | |
| VMHEALHNHYTQKSLSLSPGK | |

or the heavy-chain amino acid sequence:

| | |
|---|---|
| EVQLVESGGGLVQPGGSLRLSCAASGYTFTSYNMHWVRQAPGKGLEWVGAIYPGNGDTSY | (SEQ ID NO: 5) |
| NQKEKGRFTISVDKSKNTLYLQMNSLRAEDTAVYYCARVVYYSNSYWYFDVWGQGTLVTV | |
| SSASTKGPSVFPLAPSSKSTSGGTAALGCLVKDYFPEPVTVSWNSGALTSGVHTFPAVLQSSG | |
| LYSLSSVVTVPSSSLGTQTYICNVNHKPSNTKVDKKVEPKSCDKTHTCPPCPAPELLGGPSVF | |
| LFPPKPKDTLMISRTPEVTCVVVDVSHEDPEVKENWYVDGVEVHNAKTKPREEQYNSTYRV | |
| VSVLTVLHQDWLNGKEYKCKVSNKALPAPIEKTISKAKGQPREPQVYTLPPSREEMTKNQVS | |
| LTCLVKGFYPSDIAVEWESNGQPENNYKTTPPVLDSDGSFFLYSKLTVDKSRWQQGNVFSCS | |
| VMHEALHNHYTQKSLSLSPGK. | |

In another preferred embodiment, the intact humanized 2H7 antibody comprises the light-chain amino acid sequence:

| | |
|---|---|
| DIQMTQSPSSLSASVGDRVTITCRASSSVSYLHWYQQKPGKAPKPLIYAPSNLASGVPSRFSGS | (SEQ ID NO: 35) |
| GSGTDFTLTISSLQPEDFATYYCQQWAFNPPTFGQGTKVEIKRTVAAPSVFIFPPSDEQLKSGT | |
| ASVVCLLNNFYPREAKVQWKVDNALQSGNSQESVTEQDSKDSTYSLSSTLTLSKADYEKHK | |
| VYACEVTHQGLSSPVTKSFNRGEC | |

and the heavy-chain amino acid sequence:

| | |
|---|---|
| EVQLVESGGGLVQPGGSLRLSCAASGYTFTSYNMHWVRQAPGKGLEWVGAIYPGNGDTSY | (SEQ ID NO: 36) |
| NQKEKGRFTISVDKSKNTLYLQMNSLRAEDTAVYYCARVVYYSNSYWYFDVWGQGTLVTV | |
| SSASTKGPSVFPLAPSSKSTSGGTAALGCLVKDYFPEPVTVSWNSGALTSGVHTFPAVLQSSG | |
| LYSLSSVVTVPSSSLGTQTYICNVNHKPSNTKVDKKVEPKSCDKTHTCPPCPAPELLGGPSVF | |
| LFPPKPKDTLMISRTPEVTCVVVDVSHEDPEVKENWYVDGVEVHNAKTKPREEQYNSTYRV | |
| VSVLTVLHQDWLNGKEYKCKVSNKALPAPIEKTISKAKGQPREPQVYTLPPSREEMTKNQVS | |
| LTCLVKGFYPSDIAVEWESNGQPENNYKTTPPVLDSDGSFFLYSKLTVDKSRWQQGNVFSCS | |
| VMHEALHNHYTQKSLSLSPGK. | |

In another preferred embodiment, the humanized 2H7 antibody comprises the variable light-domain sequence of SEQ ID NO:37 and the variable heavy-domain sequence of SEQ ID NO: 18, wherein the antibody further contains an amino acid substitution of D56A in CDR H2, and N100 in CDR H3 is substituted with Y or W, wherein SEQ ID NO:37 has the sequence:

| | |
|---|---|
| DIQMTQSPSSLSASVGDRVTITCRASSSVSYLHWYQQKPGKAPKPLIYAPSNLASGVPSRFSGS | (SEQ ID NO: 37) |
| GSGTDFTLTISSLQPEDFATYYCQQWAFNPPTFGQGTKVEIKR. | |

In one embodiment of this lattermost humanized 2H7 antibody, N100 is substituted with Y. In another embodiment, N100 is substituted with W. Moreover, in a further embodiment, the antibody comprises the substitution S100aR in CDR H3, preferably further comprising at least one amino acid substitution in the Fc region that improves ADCC and/or CDC activity, such as one that comprises an IgGI Fc comprising the amino acid substitutions S298A, E333A, K334A, and K326A. Alternatively, the antibody comprises the substitution S100aR in CDR H3, preferably further comprising at least one amino acid substitution in the Fc region that improves ADCC but decreases CDC activity, such as one that comprises at least the amino acid substitution K322A, as well as one that further comprises the amino acid substitutions S298A, E333A, K334A.

In one preferred embodiment, the antibody comprises the 2H7.v511 light chain:

| | |
|---|---|
| DIQMTQSPSSLSASVGDRVTITCRASSSVSYLHWYQQKPGKAPKPLIYAPSNLASGVPSRFSGS | (SEQ ID NO: 38) |
| GSGTDFTLTISSLQPEDFATYYCQQWAFNPPTFGQGTKVEIKRTVAAPSVFIFPPSDEQLKSGT | |
| ASVVCLLNNFYPREAKVQWKVDNALQSGNSQESVTEQDSKDSTYSLSSTLTLSKADYEKHK | |
| VYACEVTHQGLSSPVTKSFNRGEC | |

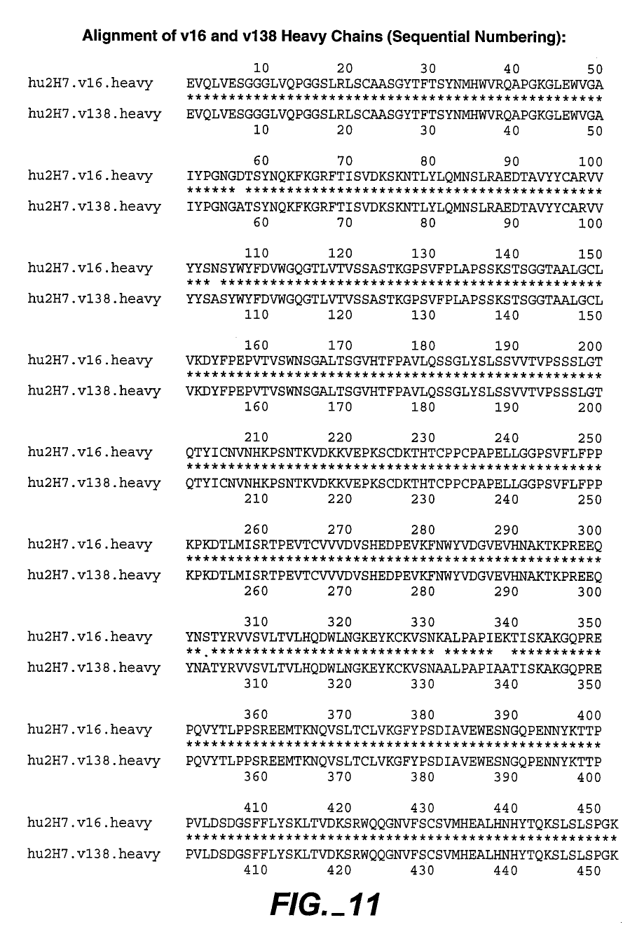

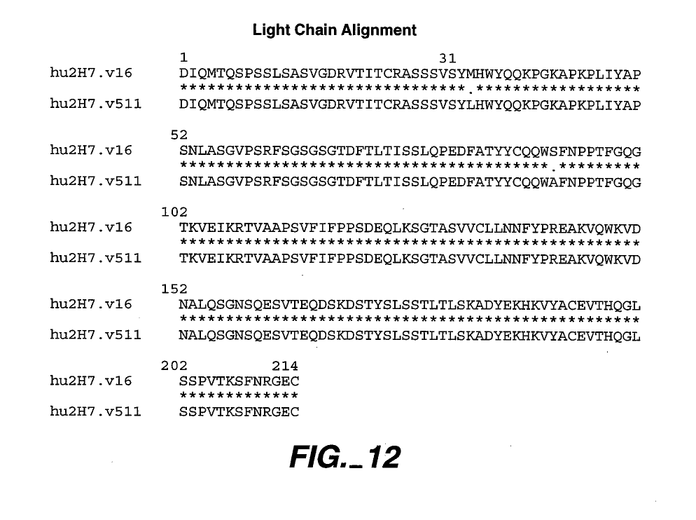

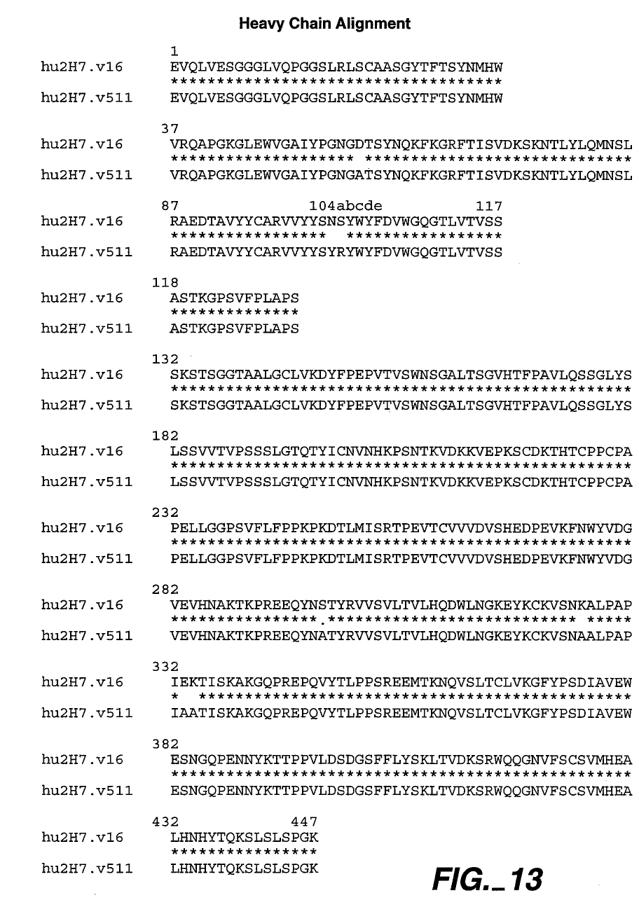

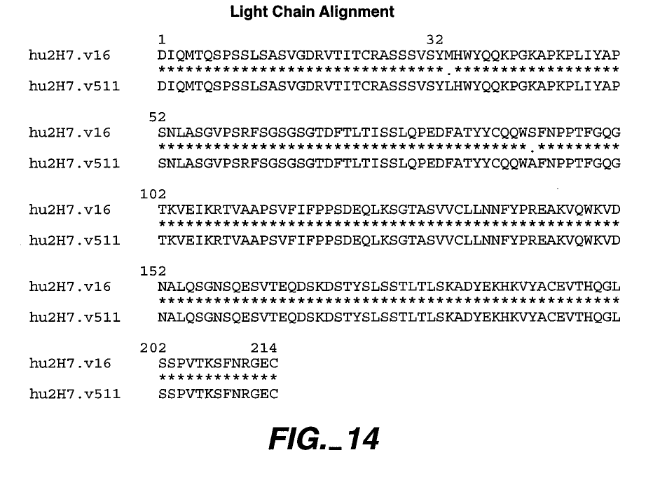

and the 2H7.v5 ll heavy chain:

| | |
|---|---|
| EVQLVESGGGLVQPGGSLRLSCAASGYTFTSYNMHWVRQAPGKGLEWVGAIYPGN | (SEQ ID NO: 39) |
| GATSYNQKFKGRFTISVDKSKNTLYLQMNSLRAEDTAVYYCARVVYYSYRYWYFDVWGQ | |
| GTLVTVSSASTKGPSVFPLAPSSKSTSGGTAALGCLVKDYFPEPVTVSWNSGALTSGVHTFPA | |
| VLQSSGLYSLSSVVTVPSSSLGTQTYICNVNHKPSNTKVDKKVEPKSCDKTHTCPPCPAPELL | |
| GGPSVFLFPPKPKDTLMISRTPEVTCVVVDVSHEDPEVKFNWYVDGVEVHNAKTKPREEQY | |
| NATYRVVSVLTVLHQDWLNGKEYKCKVSNAALPAPIAATISKAKGQPRIEPQVYTLPPSREE | |
| MTKNQVSLTCLVKGFYPSDIAVEWESNGQPENNYKTTPPVLDSDGSFFLYSKLTVDKSRWQ | |
| QGNVFSCSVMHEALHNHYTQKSLSLSPGK. | |
| See FIGS.12-15 regarding sequence alignments of these chains | |
| with those of 2H7.v16 light- and heavy-chain sequences, | |
| respectively, using EU or Kabat numbering. | |

### Example 4

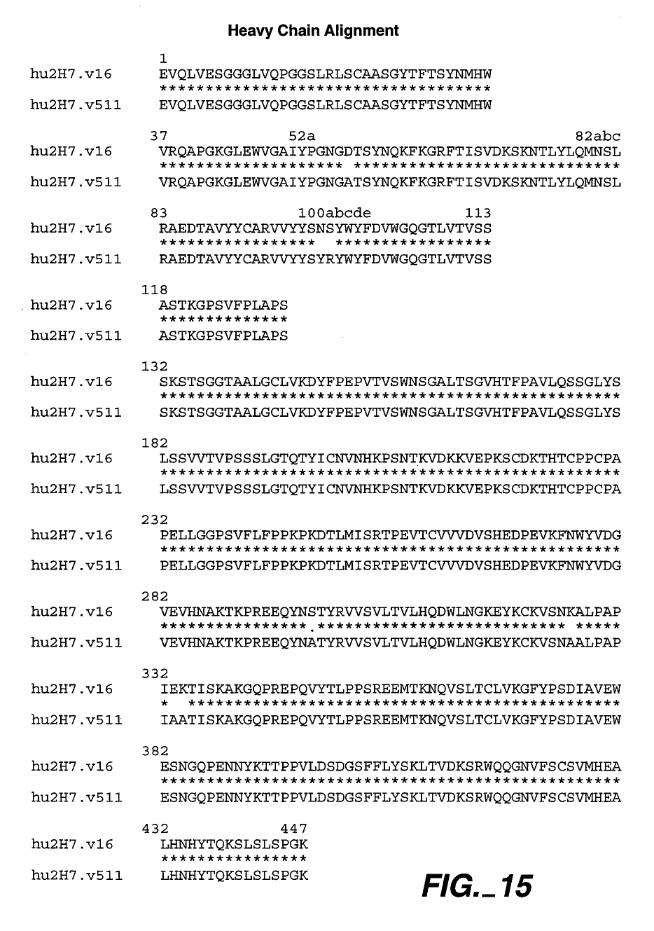

The anti-idiotypic antibody-based assay herein has been used for measuring other variants of mouse 2H7, for example, v96 and v327, in mouse serum. The typical ELISA standard curves for these experiments are shown in FIG. 16, as compared to v16. As shown in Table 2 of Example 3, in comparison to v16, v 96 has in its heavy chain D56A, N100A, and in its light chain S92A. Version 327 has, in comparison to v16, N941 in its light chain. This assay was performed as described above in Example 1 using the same anti-idiotypic antibodies as in Example 1. The standard curves shown in FIG. 16 indicate that the assay was used successfully and sensitively to measure these three antibodies in mouse serum. The ELISA for measuring mouse IgG was not performed since it would also detect endogenous mouse IgG in mouse serum.

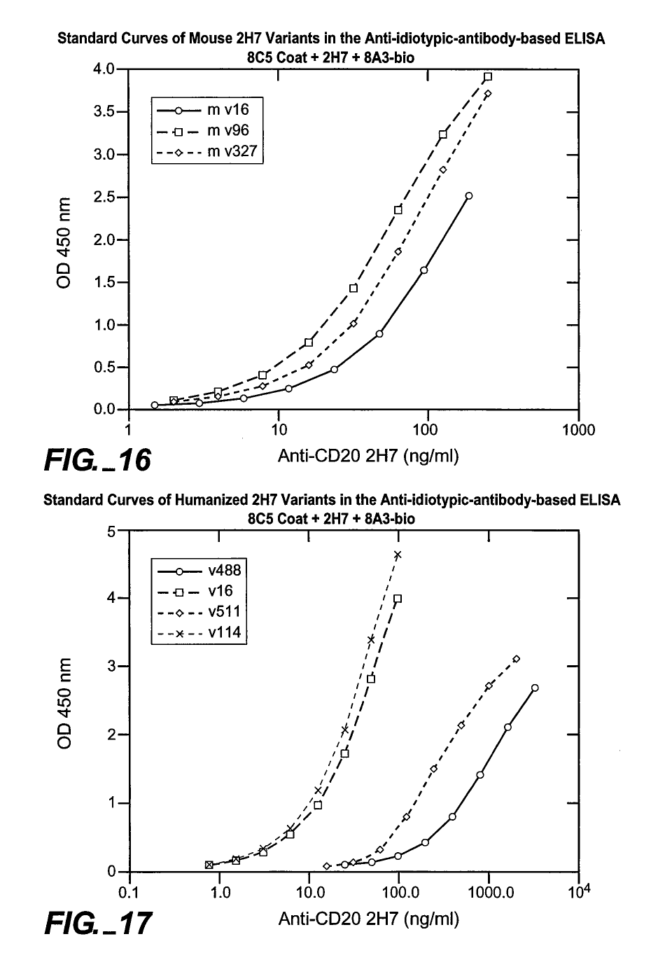

This assay was also used to measure humanized 2H7 in mouse serum. For example, humanized 2H7 variants v114 (in Table 2 of Example 3), v488 ((heavy chain: N100D, K326A, S298A, E233A, K234A versus v16), and v511 (in Table 2 of Example 3) were measured along with v16 using the assay as described in Example 1, using antibody 8C5 as coat/capture antibody and biotinylated antibody 8A3 as detection antibody. The typical ELISA standard curves for these experiments, as shown in FIG. 17, indicate that the assays for v16 and v114 were more sensitive than those for v488 and v511. For this purpose, an ELISA for measuring human IgG in mouse serum was also used in addition to the anti-idiotypic antibody-based ELISA for these latter two versions.

It is expected that the anti-idiotypic-antibody-based ELISA will be more sensitive in measuring humanized 2H7 v488 and v511 in human serum/plasma to support clinical trials using different anti-idiotypic antibodies to v 488 and v511, which can be prepared by the same or essentially the same materials and methods as in Example 1 using v488 or v511 as the antigen, respectively.

IV. Deposit of Cell Lines

The following hybridoma cell lines were deposited with the American Type Culture Collection (ATCC) located at 10801 University Boulevard, Manassas, Va. 20110-2209, U.S.A., and accorded the accession numbers:

| Hybridoma | ATCC Accession No. | Deposit Date |
|---|---|---|
| 8C5.1 | PTA-5915 | Apr. 15, 2004 |
| 8A3.10 | PTA-5914 | Apr. 15, 2004 |

(These hybridomas correspond to the clones 8C5 and 8A3, respectively.)

These deposits were made under the provisions of the Budapest Treaty on the International Recognition of the Deposit of Microorganisms for the Purpose of Patent Procedure and the Regulations thereunder (Budapest Treaty). This assures maintenance of viable cultures for 30 years from the date of deposit. The organisms will be made available by ATCC under the terms of the Budapest Treaty, and subject to an agreement between Genentech, Inc. and ATCC, which assures permanent and unrestricted availability of the progeny of the cultures to the public upon issuance of the pertinent U.S. patent or upon laying open to the public of any U.S. or foreign patent application, whichever comes first, and assures availability of the progeny to one determined by the U.S. Director of Patents and Trademarks to be entitled thereto according to 35 USC § 122 and the Director's rules pursuant thereto (including 37 CFR § 1.14 with particular reference to 886 OG 638). The assignee in the present application states that the deposits have been made under the terms of the Budapest Treaty and that subject to 37 CFR § 1.808(b), all restrictions imposed by the depositor on the availability to the public of the deposited material will be irrevocably removed upon the granting of a patent.

The assignee of the present application has agreed that if the cultures on deposit should die or be lost or destroyed when cultivated under suitable conditions, they will be promptly replaced on notification with a viable specimen of the same culture. Availability of the deposited strains is not to be construed as a license to practice the invention in contravention of the rights granted under the authority of any government in accordance with its patent laws. The making of these deposits is by no means an admission that deposits are required to enable the invention.

What is claimed is:

1. An enzyme-linked immunosorbent assay (ELISA) method for specifically detecting in a biological sample an antibody of interest that binds to a cell-surface, multi-transmembrane protein comprising an intervening extracellular domain of less than about 75 amino acids, comprising (a) contacting and incubating the biological sample with a capture reagent, wherein the capture reagent is an anti-idiotypic antibody binding to the idiotype of the antibody of interest but not to the idiotype of at least one other antibody in the sample that binds to the protein, so as to bind any of the antibody of interest present in the sample, and (b) contacting the sample, and hence any bound antibody of interest, with a detectable antibody that binds to the antibody of interest, and measuring the level of any of the antibody of interest bound to the capture reagent using a detection means for the detectable antibody.

2. The method of claim 1 wherein the antibody of interest is a monoclonal antibody.

3. The method of claim 1 wherein the antibody of interest is a humanized antibody.

4. The method of claim 1 wherein the antibody of interest is a murine antibody.

5. The method of claim 1 wherein the detectable antibody is a detectable anti-idiotypic antibody binding to the idiotype of the antibody of interest but not to the idiotype of at least one other antibody in the sample that binds to the protein.

6. The method of claim 1 wherein the biological sample is isolated from a human subject.

7. The method of claim 1 wherein the biological sample is isolated from a mouse subject.

8. The method of claim 1 wherein the measuring step further comprises using a standard curve to determine the level of the antibody of interest compared to a known level.

9. The method of claim 1 wherein the biological sample is plasma, serum, or urine.

10. The method of claim 9 wherein the sample is serum.

11. The method of claim 1 wherein the protein is CD20.

12. The method of claim 1 wherein the antibody of interest is a humanized 2H7 antibody.

13. The method of claim 12 wherein the antibody of interest is an intact antibody or antibody fragment comprising the variable light-chain sequence:

| | |
|---|---|
| DIQMTQSPSSLSASVGDRVTITCRASSSVSYMHWYQQKPGKAPKPLIYAPSNLAS | (SEQ ID NO: 1) |
| GVPSRFSGSGSGTDFTLTISSLQPEDFATYYCQQWSFNPPTFGQGTKVEIKR; | |

and the variable heavy-chain sequence:

| | |
|---|---|
| EVQLVESGGGLVQPGGSLRLSCAASGYTFTSYNMHWVRQAPGKGLEWVGAIY | (SEQ ID NO: 2) |
| PGNGDTSYNQKFKGRFTISVDKSKNTLYLQMNSLRAEDTAVYYCARVVYYSNS | |
| YWYFDVWGQGTLVTVSS. | |

14. The method of claim 12 wherein the antibody of interest is an intact antibody comprising the light-chain amino acid sequence:

| | |
|---|---|
| DIQMTQSPSSLSASVGDRVTITCRASSSVSYMHWYQQKPGKAPKPLIYAPSNLAS | (SEQ ID NO: 3) |
| GVPSRFSGSGSGTDFTLTISSLQPEDFATYYCQQWSFNPPTFGQGTKVEIKRTVA | |
| APSVFIFPPSDEQLKSGTASVVCLLNNFYPREAKVQWKVDNALQSGNSQESVTE | |
| QDSKDSTYSLSSTLTLSKADYEKHKVYACEVTHQGLSSPVTKSFNRGEC; | |

and the heavy-chain amino acid sequence:

| | |
|---|---|
| EVQLVESGGGLVQPGGSLRLSCAASGYTFTSYNMHWVRQAPGKGLEWVGAIY | (SEQ ID NO: 4) |
| PGNGDTSYNQKFKGRFTISVDKSKNTLYLQMNSLRAEDTAVYYCARVVYYSNS | |
| YWYFDVWGQGTLVTVSSASTKGPSVFPLAPSSKSTSGGTAALGCLVKDYFPEPV | |
| TVSWNSGALTSGVHTFPAVLQSSGLYSLSSVVTVPSSSLGTQTYICNVNHKPSNT | |
| KVDKKVEPKSCDKTHTCPPCPAPELLGGPSVFLFPPKPKDTLMISRTPEVTCVVV | |
| DVSHEDPEVKFNWYVDGVEVHNAKTKPREEQYNSTYRVVSVLTVLHQDWLNG | |
| KEYKCKVSNKALPAPIEKTISKAKGQPREPQVYTLPPSREEMTKNQVSLTCLVK | |
| GFYPSDIAVEWESNGQPENNYKTTPPVLDSDGSFFLYSKLTVDKSRWQQGNVFS | |
| CSVMHEALHNHYTQKSLSLSPGK. | |

15. The method of claim 12 wherein the antibody of interest is an intact antibody comprising the light-chain amino acid sequence:

| | |
|---|---|
| DIQMTQSPSSLSASVGDRVTITCRASSSVSYMHWYQQKPGKAPKPLIYAPSNLAS | (SEQ ID NO: 3) |
| GVPSRFSGSGSGTDFTLTISSLQPEDFATYYCQQWSFNPPTFGQGTKVEIKRTVA | |
| APSVFIFPPSDEQLKSGTASVVCLLNNFYPREAKVQWKVDNALQSGNSQESVTE | |
| QDSKDSTYSLSSTLTLSKADYEKHKVYACEVTHQGLSSPVTKSFNRGEC; | |

and the heavy-chain amino acid sequence:

| | |
|---|---|
| EVQLVESGGGLVQPGGSLRLSCAASGYTFTSYNMHWVRQAPGKGLEWVGAIY | (SEQ ID NO: 5) |
| PGNGDTSYNQKFKGRFTISVDKSKNTLYLQMNSLRAEDTAVYYCARVVYYSNS | |
| YWYFDVWGQGTLVTVSSASTKGPSVFPLAPSSKSTSGGTAALGCLVKDYFPEPV | |
| TVSWNSGALTSGVHTFPAVLQSSGLYSLSSVVTVPSSSLGTQTYICNVNHKPSNT | |
| KVDKKVEPKSCDKTHTCPPCPAPELLGGPSVFLFPPKPKDTLMISRTPEVTCVVV | |
| DVSHEDPEVKFNWYVDGVEVHNAKTKPREEQYNATYRVVSVLTVLHQDWLN | |
| GKEYKCKVSNKALPAPIAATISKAKGQPREPQVYTLPPSREEMTKNQVSLTCLV | |
| KGFYPSDIAVEWESNGQPENNYKTTPPVLDSDGSFFLYSKLTVDKSRWQQGNV | |
| FSCSVMHEALHNHYTQKSLSLSPGK. | |

16. The method of claim 1 wherein the capture reagent is a monoclonal antibody.

17. The method of claim 1 wherein the capture reagent is a murine antibody.

18. The method of claim 1 wherein the capture reagent is antibody 8A3 or antibody 8C5.

19. The method of claim 1 wherein the capture reagent and detectable antibody are the same.

20. The method of claim 19 wherein antibody 8A3 is used as capture reagent and detectable antibody,

21. The method of claim 1 wherein the capture reagent and detectable antibody are different.

22. The method of claim 21 wherein antibody 8C5 is used as capture reagent and antibody 8A3 is used as detectable antibody.

23. The method of claim 1 comprising the steps of: (a) contacting and incubating the biological sample with the capture reagent immobilized to a solid support so as to bind any of the antibody of interest present in the sample with the capture reagent; (b) separating the biological sample from the immobilized capture reagent bound to any of the antibody of interest present; (c) contacting the immobilized capture reagent bound to any of the antibody of interest present with a detectable anti-idiotypic antibody against the antibody of interest, said detectable antibody binding to the idiotype of the antibody of interest but not to the idiotype of at least one other antibody in the sample that binds to the protein; and (d) measuring the level of any of the antibody of interest bound to the capture reagent using a detection means for the detectable antibody.

24. The method of claim 23 wherein the immobilized capture reagent is coated on a microtiter plate.

25. The method of claim 23 wherein the detectable antibody is directly detectable.

26. The method of claim 25 wherein the detectable antibody is amplified by a fluorimetric or colorimetric reagent.

27. The method of claim 25 wherein the detectable antibody is biotinylated and the detection means is avidin or streptavidin-β-horseradish peroxidase.

28. The method of claim 1 that is cell based.

29. An antibody 8A3 comprising SEQ ID NOS:7 and 9 for the heavy and light chains, respectively, and obtainable from hybridoma 8A3.10 deposited under ATCC number PTA-5914.

30. The antibody of claim 29 conjugated to a detectable label.

31. An antibody 8C5 obtainable from hybridoma 8C5.1 deposited under ATCC number PTA-5915.

32. The antibody of claim 31 conjugated to a detectable label.

33. A hybridoma 8C5.1 or 8A3.10 deposited under ATCC deposit number PTA-5915 or PTA-5914, respectively.

34. An immunoassay kit for specifically detecting in a biological sample an antibody of interest that binds to a cell-surface, multi-transmembrane protein comprising an intervening extracellular domain of less than about 75 amino acids, the kit comprising: (a) a container containing, as a capture reagent, an anti-idiotypic antibody binding to the idiotype of the antibody of interest but not to the idiotype of at least one other antibody in the sample that binds to the protein; (b) a container containing a detectable anti-idiotypic antibody that binds to the idiotype of the antibody of interest but not to the idiotype of at least one other antibody in the sample that binds to the protein; and (c) instructions for detecting said antibody of interest.

35. The kit of claim 34 useful in an ELISA method for detecting the antibody of interest.

36. The kit of claim 34 further comprising a solid support for the capture reagent.

37. The kit of claim 34 wherein the capture reagent is immobilized on the solid support.

38. The kit of claim 34 wherein the capture reagent is coated on a microtiter plate.

39. The kit of claim 34 further comprising a detection means for the detectable antibody.

40. The kit of claim 39 wherein the detection means is avidin or streptavidin-horseradish peroxidase.

41. The kit of claim 34 further comprising purified antibody of interest as a standard.

42. The kit of claim 34 wherein the capture reagent and detectable antibody are monoclonal antibodies.

43. The kit of claim 34 wherein the capture reagent and detectable antibody are the same.

44. The kit of claim 34 wherein the capture reagent and detectable antibody are different.

45. The kit of claim 34 wherein the protein is CD20.

46. The kit of claim 34 wherein the antibody of interest is a humanized antibody.

47. The kit of claim 34 wherein the antibody of interest is a humanized 2H7 antibody.
